(19) United States
(12) Patent Application Publication
(10) Pub. No.: US 2015/0119633 A1
(43) Pub. Date: Apr. 30, 2015

(54) INTRAVASCULAR DEVICE ATTACHMENT SYSTEM HAVING BIOLOGICAL MATERIAL

(71) Applicant: COOK MEDICAL TECHNOLOGIES LLC, Bloomington, IN (US)

(72) Inventors: Kenneth Haselby, Battle Ground, IN (US); Brian D. Choules, Lafayette, IN (US); Blayne A. Roeder, Lafayette, IN (US); Richard B. Sisken, West Lafayette, IN (US); David Eric Orr, Piedmont, SC (US); James D. Purdy, Lafayette, IN (US); Steven J. Charlebois, West Lafayette, IN (US); David D. Grewe, West Lafayette, IN (US); Neal E. Fearnot, West Lafayette, IN (US); Alan R. Leewood, Lafayette, IN (US)

(73) Assignee: Cook Medical Technologies LLC

(21) Appl. No.: 14/568,448

(22) Filed: Dec. 12, 2014

Related U.S. Application Data
(63) Continuation of application No. 12/863,038, filed on Jan. 19, 2011.
(60) Provisional application No. 61/022,068, filed on Jan. 18, 2008.

(51) Int. Cl. A61M 1/12

(52) U.S. Cl. CPC A61M 1/125
USPC 600/16

(57) ABSTRACT

A method of attaching an intravascular device to a vessel wall of a body vessel is disclosed. The attachment system includes an intravascular device and biological attachment material connected to the intravascular device. The biological attachment material is configured to attach the intravascular device to the vessel wall.

### CROSS-REFERENCE TO RELATED APPLICATIONS

This application is a continuation of co-pending U.S. patent application Ser. No. 12/863,038, entitled “Intravascular Device Attachment System Having Biological Material,” filed on Jul. 15, 2010, which application is the U.S. National Stage of International Application No. PCT/US2009/031245, entitled “Intravascular Device Attachment System Having Biological Material,” filed on Jan. 16, 2009, which claims the benefit of U.S. Provisional Application No. 61/022,068, filed on Jan. 18, 2008, entitled “Intravascular Device Attachment System”, each of which is incorporated herein by reference in its entirety.

This application is related to U.S. patent application Ser. No. 12/863,030, entitled “Intravascular Device Attachment System Having Tubular Expandable Body,” filed on Jul. 15, 2010, and published on May 5, 2011 as U.S. Publication No. 2011/0106120, which is the U.S. National Stage of International Application No. PCT/US2009/031232, filed on Jan. 16, 2009, and U.S. patent application Ser. No. 12/863,034, entitled “Intravascular Device Attachment System Having Biological Material,” filed Jul. 15, 2010, and published on May 5, 2011 as U.S. Publication No. 2011/0106115, which is the U.S. National Stage of International Application No. PCT/2009/031236, filed on Jan. 16, 2009, the contents of which are incorporated herein by reference in their entirety.

### BACKGROUND OF THE INVENTION

1. Field of the Invention

The present invention generally relates to medical devices. More particularly, the invention relates to medical devices that include systems for attaching intravascular devices to body vessels.

2. Description of Related Art

Functional intravascular devices that may be implanted within a vascular system are becoming increasingly used by physicians. These types of implantable intravascular devices may include, but are not limited to, heart pumps, ventricular assist devices, active and passive drug eluting cartridges, valves, and sensors and other instrumentation.

It may be desirable for some functional intravascular devices to remain implanted in a patient for extended periods of time. Furthermore, it may be desirable for such functional intravascular devices to be implanted through minimally invasive methods of implantation, and therefore, to be implanted percutaneously. Often times, it may be desirable to implant the devices in a retrievable manner, or in a manner that causes little trauma to the blood vessel. Thus, an invasive attachment procedure, such as one involving anastomosis between the intravascular device and the body vessel, may be undesirable. Furthermore, some intravascular devices create a force tending to cause migration, and therefore, the attachment system must be able to withstand such forces. There may be trade-offs between designing an attachment system having a strong attachment that is minimally invasive, retrievable, and percutaneously deliverable.

In view of the above, there exists a need for an improved attachment system for a secure intravascular device attachment system that is minimally invasive, while allowing for percutaneous placement of the intravascular device.

### BRIEF SUMMARY OF THE INVENTION

In satisfying the above need, as well as overcoming the numerous drawbacks in the prior art, the present invention provides a secure attachment system for an intravascular device, which is minimally invasive and allows for percutaneous placement of the device, if desired.

In one aspect, the present invention provides an intravascular device attachment system for securing an intravascular device to a vessel wall. The attachment system includes an intravascular device and a plurality of struts extending therefrom. The intravascular device has a first end and a second end, the intravascular device defining a longitudinal axis along a length thereof. Each strut has an attached end connected to one of the first and second ends of the intravascular device, and each strut is configured to move along a strut path relative to the longitudinal axis between an expanded state for engaging with the vessel wall and a collapsed state for delivery or retrieval. At least some of the struts have a free end configured to engage the vessel wall in the expanded state.

In another aspect, a first plurality of struts is connected to a first end of the intravascular device and a second plurality of struts is connected to the second end of the intravascular device.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1A is a side view of an attachment system according to the principles of the present invention;

FIG. 1B is an end view of the attachment system of FIG. 1A, in accordance with the principles of the present invention;

FIG. 2A is a side view of a delivery and retrieval assembly for use with the attachment system of FIGS. 1A and 1B, in accordance with the principles of the present invention;

FIG. 2B is an exploded view of the delivery and retrieval assembly of FIG. 2A, in accordance with the principles of the present invention;

FIG. 3 is a side sectional view of a portion of the delivery and retrieval system of FIGS. 2A and 2B, in accordance with the principles of the present invention;

FIG. 4 is a side sectional view of a body vessel including the attachment system of FIGS. 1A and 1B and a portion of the delivery and retrieval system of FIGS. 2A-3, in accordance with the principles of the present invention;

FIG. 5 is a side sectional view of a body vessel including the attachment system of FIGS. 1A, 1B, and 4, in accordance with the principles of the present invention;

FIG. 6A is a side view of another attachment system according to the principles of the present invention;

FIG. 6B is an end view of the attachment system of FIG. 6A, in accordance with the principles of the present invention;

FIG. 7 is an end sectional view of a body vessel including yet another attachment system according to the principles of the present invention;

FIG. 8 is an end sectional view of a body vessel including still another attachment system according to the principles of the present invention;

FIG. 9A is an end sectional view of a body vessel including still another attachment system according to the principles of the present invention;

FIG. 9B is a side sectional view of a body vessel including the attachment system of FIG. 9A, in accordance with the principles of the present invention;

FIG. 10A is a side sectional view of a body vessel including still another attachment system according to the principles of the present invention, the attachment system being shown in a partially cut away side view;

FIG. 10B is an end sectional view of a body vessel including the attachment system of FIG. 10A, in accordance with the principles of the present invention;

FIG. 11A is a side sectional view of a body vessel including still another attachment system according the principles of the present invention;

FIG. 11B is a side sectional view of a body vessel including the attachment system of FIG. 11A, wherein the attachment system is undergoing a staged delivery, in accordance with the principles of the present invention;

FIG. 12A is a side sectional view of a body vessel including still another attachment system according to the principles of the present invention;

FIG. 12B is a side sectional view of a body vessel including the attachment system of FIG. 12A, wherein a restricting member of the attachment system is partially removed, in accordance with the principles of the present invention;

FIG. 12C is a side sectional view of a body vessel including the attachment system of FIGS. 12A and 12B, wherein the restricting member is further removed, in accordance with the principles of the present invention;

FIG. 12D is a side sectional view of a body vessel including the attachment system of FIGS. 12A-12C, the attachment system being removed from the vessel wall in accordance with the principles of the present invention;

FIG. 13 is a side sectional view of a body vessel including still another attachment system according to the principles of the present invention;

FIG. 14 is a side sectional view of a body vessel including still another attachment system according to the principles of the present invention;

FIG. 15A is a side sectional view of a body vessel including still another attachment system according to the principles of the present invention;

FIG. 15B is a side sectional view of a body vessel including the attachment system of FIG. 15A, the attachment system being partially removed from the vessel wall in accordance with the principles of the present invention;

FIG. 15C is a side sectional view of a body vessel including the attachment system of FIGS. 15A and 15B, the attachment system being removed from the vessel wall in accordance with the principles of the present invention;

FIG. 16A is a side view of still another attachment system according to the principles of the present invention;

FIG. 16B is an end view of the attachment system of FIG. 16A, in accordance with the principles of the present invention;

FIG. 17A is a side view of still another attachment system according to the principles of the present invention;

FIG. 17B is an end view of the attachment system of FIG. 17A, in accordance with the principles of the present invention;

FIG. 18A is a side sectional view of a cannula sheath including still another attachment system in a collapsed state according to the principles of the present invention;

FIG. 18B is a side sectional view of a cannula sheath including the attachment system of FIG. 18A in the collapsed state, the attachment system being partially deployed from the cannula sheath, in accordance with the principles of the present invention;

FIG. 18C is a side sectional view of a body vessel including the attachment system of FIGS. 18A and 18B in an expanded state, according to the principles of the present invention;

FIG. 18D is an end sectional view of a body vessel including the attachment system of FIGS. 18A-18C in an expanded state, according to the principles of the present invention;

FIG. 19 is a perspective view of still another attachment system according to the principles of the present invention;

FIG. 20 is a perspective view of still another attachment system according to the principles of the present invention;

FIG. 21A is a side view of still another attachment system according to the principles of the present invention;

FIG. 21B is an end view of the attachment system of FIG. 21A, in accordance with the principles of the present invention;

FIG. 22 is a perspective view of still another attachment system according to the principles of the present invention;

FIG. 23 is a perspective view of still another attachment system according to the principles of the present invention;

FIG. 24 is a perspective view of an intravascular device and a key for use with an attachment system according to the principles of the present invention;

FIG. 25 is a side view of still another attachment system according to the principles of the present invention;

FIG. 26A is an end sectional view of a body vessel including still another attachment system according to the principles of the present invention;

FIG. 26B is a side sectional view of a body vessel including the attachment system of FIG. 26A, in accordance with the principle of the present invention;

FIG. 27A is a side view of still another attachment system according to the principles of the present invention;

FIG. 27B is a side view of another intravascular device for use with an attachment system according to the principles of the present invention, the intravascular device being particularly useful with the attachment system of FIG. 27A;

FIG. 28 is a side sectional view of a body vessel including still another attachment system according to the principles of the present invention;

FIG. 29A is a side sectional view of a body vessel including still another attachment system according to the principles of the present invention;

FIG. 29B is an end sectional view of a body vessel including the attachment system of FIG. 29A, in accordance with the principles of the present invention;

FIG. 30A is a side sectional view of a body vessel including still another attachment system according to the principles of the present invention; and

FIG. 30B is an end sectional view of a body vessel including the attachment system of FIG. 30A, in accordance with the principles of the present invention.

### DETAILED DESCRIPTION OF THE INVENTION

The terms “about” or “substantially” used herein with reference to a quantity includes variations in the recited quantity that are equivalent to the quantity recited, such as an amount that is insubstantially different from a recited quantity for an intended purpose or function.

The present invention generally provides an attachment system for attaching an intravascular device within a body vessel. The device is preferably delivered percutaneously. The various embodiments of the present invention provide resistance to migration of the intravascular device, while causing little trauma to the body vessel. In addition, with some embodiments, the attachment systems allow the intravascular device to be retrieved percutaneously, and in some cases, the attachment system itself to be retrieved percutaneously.

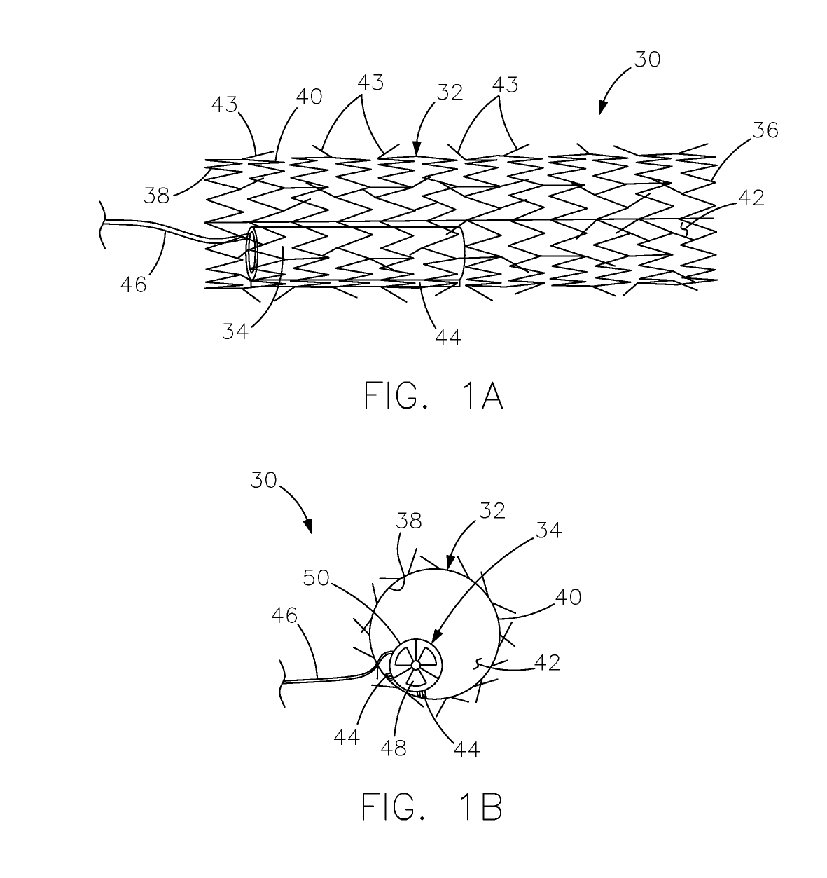

Referring now to FIGS. 1A and 1B, an attachment system for attaching an intravascular device to a vessel wall of a body vessel, which embodies the principles of the present invention, is illustrated therein and generally designated at 30. The attachment system 30 includes a tubular expandable body 32 and an intravascular device 34 attached to the tubular expandable body 32.

The tubular expandable body 32 of the attachment system 30 may resemble a stent, as shown in FIGS. 1A and 1B, wherein the tubular expandable body 32 comprises a frame having a plurality of members 36, such as wires, that are interconnected and configured to expand into an open configuration and are collapsible into a collapsed configuration. The members 36 of the frame define an interior side 38 and an exterior side 40, the interior side 38 defining a lumen 42 through the tubular expandable body 32. Preferably, the tubular expandable body 32 is cylindrical, although other configurations may be used, without falling beyond the spirit and scope of the present invention. Although the members 36 of the tubular expandable body 32 are shown having zigzag shapes, many other configurations may be suitable, such as those disclosed in U.S. Pat. No. 4,580,568; U.S. Pat. No. 5,035,706; U.S. Pat. No. 5,507,767; and U.S. Pat. No. 6,042,606, all of which are incorporated herein by reference in their entireties. For example, the members 36 could alternatively have a sinusoidal shape or a criss-cross pattern. The tubular expandable body 32 could be formed in different ways, which also affects its configuration. For example, the tubular expandable body 32 could be cut from a thin solid tube, such that it expands to a much larger tube having a lumen 42 formed therethrough. In such a configuration, the tubular expandable body 32 is collapsible down to nearly the size of the original thin solid tube that it was formed from. In the alterative, the tubular expandable body 32 could be formed from a plurality of braided members.

The tubular expandable body 32 may be made of any suitable material, for example, a superelastic material, a nickel-based superalloy, stainless steel wire, cobaltchromium-nickel-molybdenum-iron alloy, cobalt chrome-alloy, stress relieved metal (e.g., platinum), or nickel-based superalloys, such as Inconel. The tubular expandable body 32 may preferably be formed of any appropriate material that will result in a self-expanding device capable of being percutaneously inserted and deployed within a body cavity, such as shape memory material. Shape memory materials or alloys have the desirable property of becoming rigid, i.e., returning to a remembered state, when heated above a transition temperature. A shape memory alloy suitable for the present invention is nickel-titanium (NiTi) available under the more commonly known name Nitinol. When this material is heated above the transition temperature, the material undergoes a phase transformation from martensite to austenite, such that the material returns to its remembered state. The transition temperature is dependent on the relative proportions of the alloying elements Ni and Ti and the optional inclusion of alloying additives. The Nitinol could be of various types, such as linear elastic Nitinol or radiopaque Nitinol. In some embodiments, the tubular expandable body 32 could be covered, such as with a biocompatible urethane.

In one embodiment, the tubular expandable body 32 is made from Nitinol with a transition temperature that is slightly below normal body temperature of humans, which is about 98.6° F. Thus, when the tubular expandable body 32 is deployed in a body vessel and exposed to normal body temperature, the alloy of the tubular expandable body 32 will transform to austenite, that is, the remembered state, which for one embodiment of the present invention is the expanded state when the attachment system 30 is deployed in the body vessel. To remove the attachment system 30, the tubular expandable body 32 is cooled to transform the material to martensite which is more ductile than austenite, making the tubular expandable body 32 more malleable. As such, the attachment system 30 can be more easily collapsed and pulled into a lumen of a catheter for removal.

In another embodiment, the tubular expandable body 32 is made from Nitinol with a transition temperature that is above normal body temperature of humans, which is about 98.6° F. Thus, when the attachment system 30 is deployed in a body vessel and exposed to normal body temperature, the tubular expandable body 32 is in the martensitic state so that the tubular expandable body 32 is sufficiently ductile to bend or form into a desired shape, which for the present embodiment is the expanded state. To remove the attachment system 30, the tubular expandable body 32 is heated to transform the alloy of the tubular expandable body 32 to austenite so that it becomes rigid and returns to a remembered state, which for the tubular expandable body 32 is a collapsed state.

The tubular expandable body 32 is configured to move between an expanded state for attaching to a wall of a body vessel and a collapsed state for delivery or retrieval of the attachment system. The tubular expandable body 32 is configured to open radially to define the expanded state and to collapse along a central longitudinal axis, which extends through the lumen 42, to define the collapsed state. The tubular expandable body 32 has a collapsed diameter in the collapsed state and an expanded diameter in the expanded state, the expanded diameter being larger than the collapsed diameter. The tubular expandable body 32 is configured to contact the vessel wall in the expanded state along the length of the tubular expandable body 32 when deployed in the body vessel. The tubular expandable body 32 may be self-expanding, for example, through a spring force contained in its members 36, or it may be expanded through assistance, such as through the use of an inflatable balloon.

In some embodiments, the tubular expandable body 32 could have a plurality of exterior portions bent outward to contact the vessel wall in the expanded state, for example, a plurality of hooks or barbs 43, to aid in anchoring the tubular expandable body 32 to a vessel wall. The plurality of hooks or barbs 43 could alternatively be attached to the exterior side 40 of the tubular expandable body 32. The exterior portions could be bent outward, heated outward, or otherwise formed to contact the vessel wall in a manner more secure than merely contacting the vessel wall due to radial force. It should be understood that any other suitable anchoring members could alternatively or additionally be used.

In the alternative, it should be understood that the tubular expandable body 32 described herein could be configured to contact the vessel wall to secure the intravascular device 34 to the vessel wall by simply configuring the tubular expandable body 32 to provide a radial force to press the tubular expandable body 32 into the vessel wall.

As stated above, the intravascular device 34 is attached to the tubular expandable body 32. In the embodiment of FIGS. 1A and 1B, the intravascular device 34 is attached to the interior side 38 of the tubular expandable body 32, although other configurations are contemplated herein, some of which will be described in further detail below. The intravascular device 34 is preferably attached to the tubular expandable body 32 with a sacrificial or biodegradable connection 44. Such biodegradable connections could be made of magnesium alloys, silver solder, degradable polymers, degradable sutures, or any other suitable degradable connection. In FIGS. 1A and 1B, the biodegradable connection 44 is shown as a biodegradable weld, which preferably comprises a magnesium alloy or a silver solder. The biodegradable connection could be configured to wear away after a certain period of time of being in contact with a body fluid, such as blood, or it could be configured to simply wear away over time. Preferably, the biodegradable connection 44 would wear partially away over time, such that when a physician desired to remove the intravascular device 34, the physician could merely supply a small force to break the intravascular device 34 away from the tubular expandable body 32 to remove the intravascular device 34 from the tubular expandable body 32. The tubular expandable body 32 could then be left in place to serve as a stent, or in the alternative, it could be removed in any suitable manner.

To facilitate removal of the intravascular device 34, the device 34 could further comprise a cord or tether 46, or any other suitable retrieval member which could be grasped and tugged to move the intravascular device 34 into a catheter for removal. The tether 46 could be a cord that the intravascular device 34 uses for other purposes, such as a power cord or electrical conduit for a pump or a drug delivery channel for a drug eluting device. In the alternative, the tether 46 could exist merely for removal purposes. In some cases, the tether 46 or electrical feed attached to the intravascular device 34 could also serve as a strut or struts to attach the intravascular device 34 to the tubular expandable body 32.

The intravascular device 34 could be any functional device that a patient desires to have implanted in a body vessel. For example, the intravascular device 34 could be a cardiac assist device, such as a heart pump, a ventricular assist device, or any other cardiac assist device, an active and/or passive drug eluting cartridge, a valve, such as an arterial or venous valve, or a sensor or other instrumentation, such as a dissolved oxygen sensor, a dissolved carbon dioxide sensor, a pH sensor, a blood cell count sensor, or a fluid flow rate sensor. When used as a cardiac assist device, the intravascular device 34 could be placed within the venous system to supplement or compliment peripheral venousfunction. Alternatively, the cardiac assist device could be placed in the heart, in the aorta, or in another artery. When used as a sensor, the intravascular device 34 could provide a sensory feedback loop, and/or it could monitor blood conditions such as pH, flow rate, hematocrit, pressure, or any other measurable parameter. Furthermore, the intravascular device 34 could work in conjunction with adjunct devices including securement systems and auxiliary systems. In FIG. 1B, the intravascular device 34 is shown as a cardiac assist device for assisting with blood circulation, which has a pump 48 surrounded by a pump housing 50.

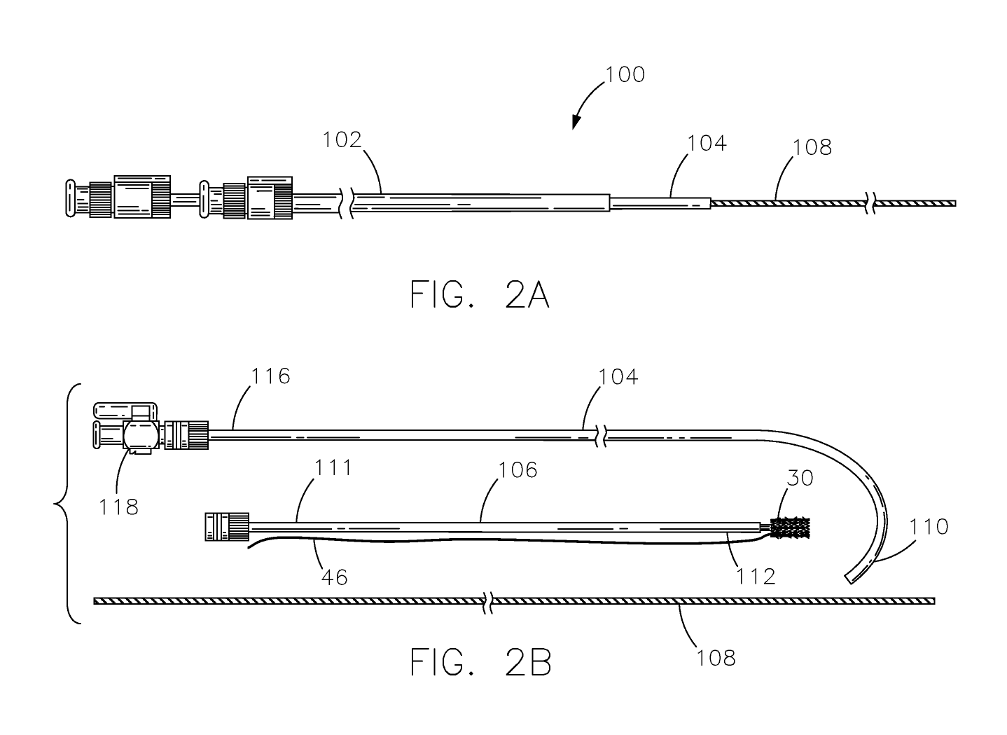

FIGS. 2A and 2B depict a delivery assembly 100 for introducing and retrieving the attachment system 30 in accordance with another embodiment of the present invention. Although described with reference to the attachment system 30 shown in FIGS. 1A and 1B, it should be understood that the delivery assembly 100 could also be used for the various attachments described below. As shown, the delivery assembly 100 includes a polytetrafluoroethylene (PTFE) introducer sheath 102 for percutaneously introducing an outer sheath 104 into a body vessel. Of course, any other suitable material for the introducer sheath 102 may be used without falling beyond the scope or spirit of the present invention. The introducer sheath 102 may have any suitable size, for example, between about three-french to eight-french. The introducer sheath 102 serves to allow the outer sheath 104 and an inner member or catheter 106 to be percutaneously inserted to a desired location in the body vessel. The inner member may also include, for example, a stylet. The introducer sheath 102 receives the outer sheath 104 and provides stability to the outer sheath 104 at a desired location of the body vessel. For example, the introducer sheath 102 is held stationary within a common visceral artery, and adds stability to the outer sheath 104, as the outer sheath 104 is advanced through the introducer sheath 102 to an area in the vasculature. The outer sheath 104 has a body extending from a proximal end 116 to a distal end 110, the body being tubular and including a sheath lumen extending therethrough.

As shown, the assembly 100 may also include a wire guide 108 configured to be percutaneously inserted within the vasculature to guide the outer sheath 104 to the desired area. The wire guide 108 provides the outer sheath 104 with a path to follow as it is advanced within the body vessel. The size of the wire guide 108 is based on the inside diameter of the outer sheath 104 and the diameter of the target body vessel.

When the distal end 110 of the outer sheath 104 is at the desired location in the body vessel, the wire guide 108 is removed and the attachment system 30, having a proximal segment contacting a distal portion 112 of the inner catheter 106, is inserted into the outer sheath 104. The inner catheter 106 is advanced through the outer sheath 104 for deployment of the attachment system 30 through the distal end 110. The catheter 106 extends from a proximal portion 111 to a distal portion 112 and is configured for axial movement relative to the outer sheath 104. In this example, the distal portion 112 is shown adjacent to the attachment system 30. Thus, before deployment, the attachment system 30 is coaxially disposed within the lumen of the outer sheath 104 and removably coupled to the distal portion 112 of the catheter 106, or in the alternative, the attachment system 30 is merely pushed by, but not coupled to, the distal portion 112 of the catheter 106.

The outer sheath 104 further has a proximal end 116 and a hub 118 to receive the inner catheter 106 and attachment system 30 to be advanced therethrough. The size of the outer sheath 104 is based on the size of the body vessel in which it percutaneously inserts, and the size of the attachment system 30 and the intravascular device 34.

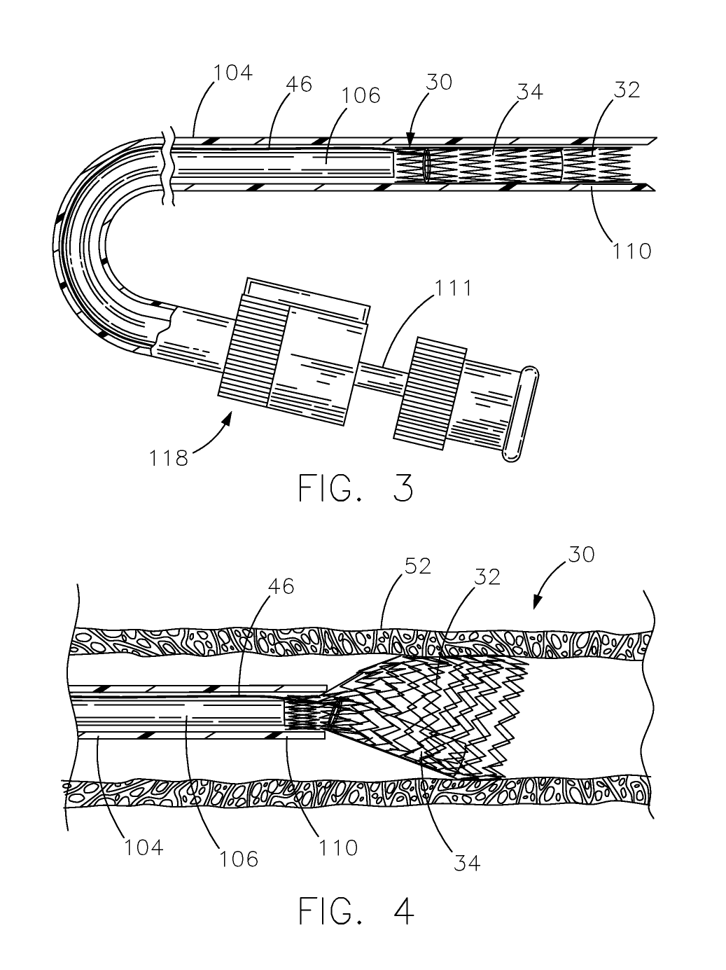

In this embodiment, and with reference to FIG. 3, the attachment system 30 and inner catheter 106 are coaxially advanced through the outer sheath 104, following removal of the wire guide 108, in order to position the attachment system 30 in the body vessel. The attachment system 30 is guided through the outer sheath 104 by the inner catheter 106, preferably from the hub 118, and exits from the distal end 110 of the outer sheath 104 at a location within the vasculature where implantation is desired. Thus, the attachment system 30 is deployable through the distal end 110 of the outer sheath 104 by means of axial relative movement of the catheter 106. In order to more easily deploy the attachment system 30 into the body vessel, the tubular expandable body 32 may have a slippery coating, such as silicone or slipcoating.

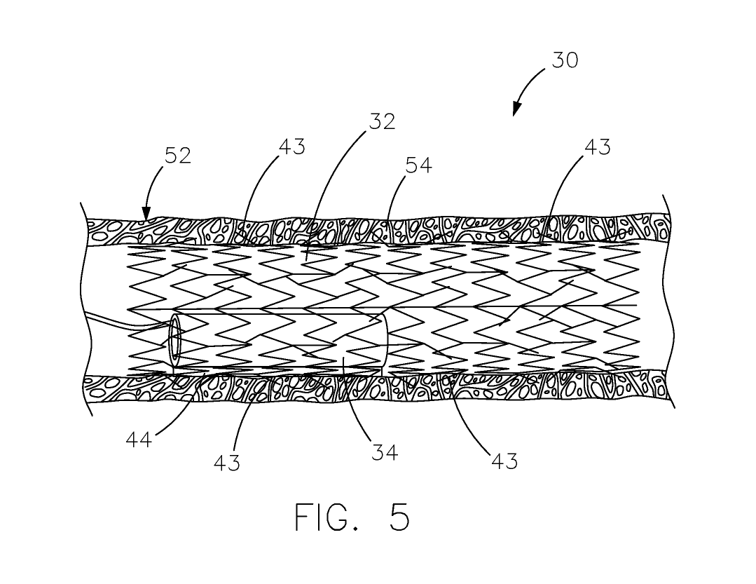

FIG. 4 illustrates the attachment system 30 being partially deployed in a body vessel 52. The attachment system 30 is being pushed from the distal end 110 of the outer sheath 104 by the catheter 106. Upon exiting the distal end 110 of the outer sheath 104, the tubular expandable body 32 moves from the collapsed state to the expanded state. FIG. 5 illustrates the attachment system 30 being fully deployed within the body vessel 52. The tubular expandable body 32 is shown in the expanded state and contacting the wall 54 of the body vessel 52. In this embodiment, the tubular expandable body 32 automatically expands into the expanded state by virtue of a radial spring force in the members 36 of the tubular expandable body 32. The barbs 43 of the tubular expandable body 32 anchor into the wall 54 of the body vessel 52 to further secure the attachment system 30 to the vessel wall 54.

Likewise, the delivery and retrieval system 100 (FIGS. 2A and 2B) may also retrieve the attachment system 30 by positioning the distal end 110 of the outer sheath 104 adjacent the deployed attachment system 30 in the vasculature. The inner catheter 106 is advanced through the outer sheath 104 until the distal portion 112 protrudes from the distal end 110 of the outer sheath 104. The distal portion 112 is preferably coupled to a proximal end of the intravascular device 34 or the tubular expandable body 32, after which the inner catheter 106 is retracted proximally, drawing the intravascular device 34 or the attachment system 30 into the outer sheath 104. The intravascular device 34 may be easily found and grasped if the tether 46 is used as a guide. In some embodiments, the tether 46 could be used to pull the intravascular device 34 into and through the outer sheath 106, such that the catheter 106 is not needed for retrieval.

It is understood that the assembly described above is merely one example of an assembly that may be used to deploy the attachment system 30 in a body vessel. Of course, other apparatus, assemblies and systems may be used to deploy any embodiment of the attachment system 30 without falling beyond the scope or spirit of the present invention.

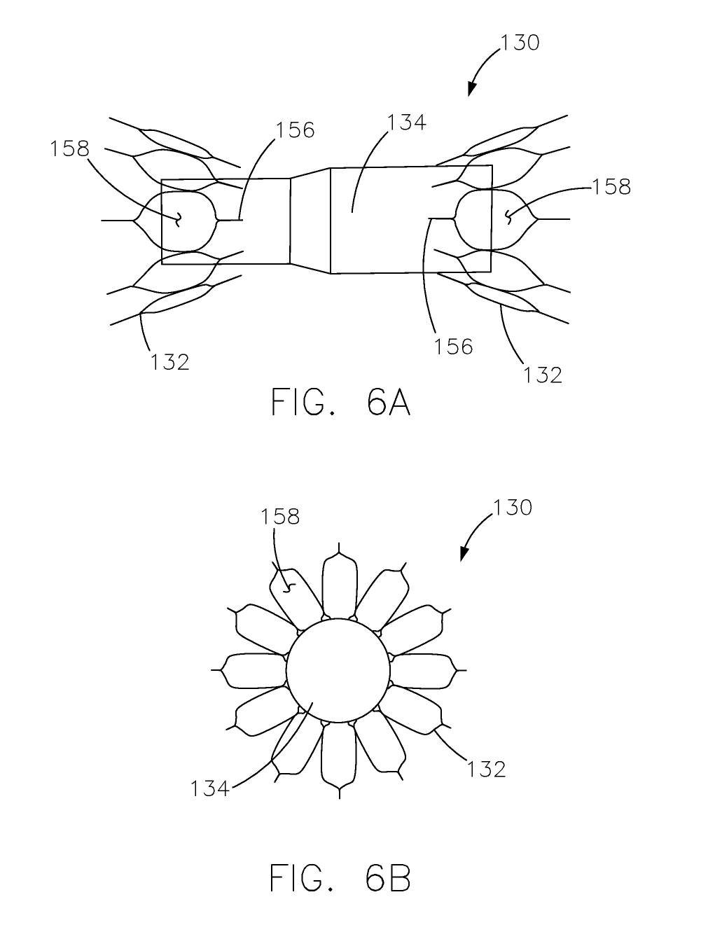

Now with reference to FIGS. 6A and 6B, another attachment system 130 for attaching an intravascular device 134 to a vessel wall of a body vessel (not shown) is illustrated. The attachment system 130 includes an intravascular device 134 attached to a pair of tubular expandable bodies 132. The tubular expandable bodies 132 are connected to the intravascular device 134 preferably with a biodegradable connection, as hereinbefore described, although it is also contemplated that a non-biodegradable connection could be used, such as laser welding or non-biodegradable sutures. Each of the tubular expandable bodies 132 may be similar to the tubular expandable body 32 of FIGS. 1A and 1B, for example, each tubular expandable body 132 may be collapsible and expandable, and each may be comprised of the various materials mentioned above. The tubular expandable bodies 132 are attached to the intravascular device 134 at an end 156 of each tubular expandable body 132, such that the remainder of each tubular expandable body 132 extends radially outward to contact a vessel wall (not shown). With reference to FIG. 6B, it may be seen that fluid may flow around the intravascular device 134 through the plurality of openings 158 in each tubular expandable body 132. In all other respects, the attachment system 130 may be similar to the attachment system 30 of FIGS. 1A and 1B, and it may be deployable and retrievable as hereinbefore described.

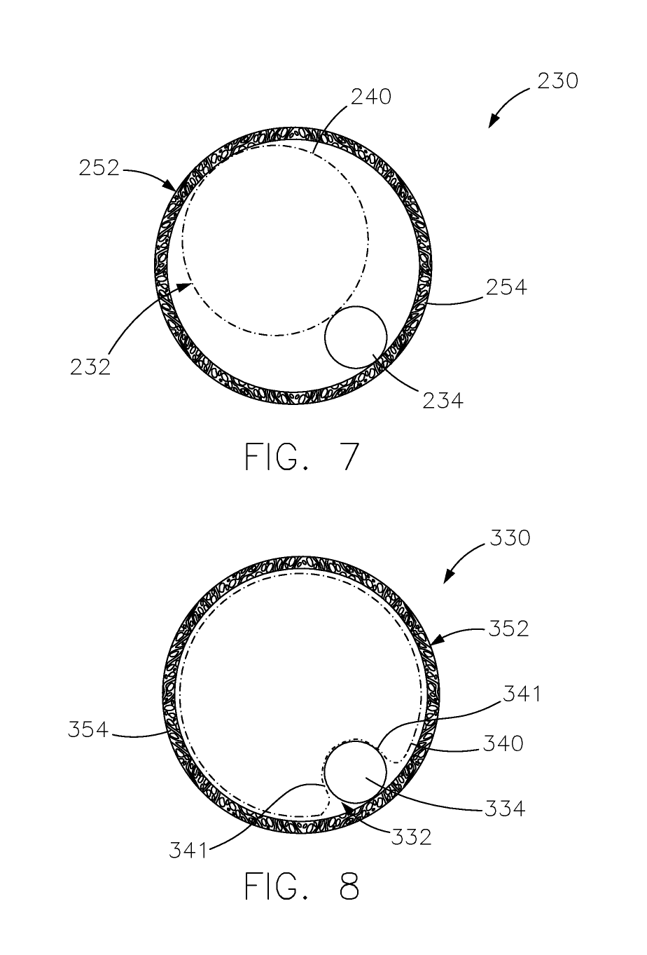

Now with reference to FIG. 7, another attachment system 230 for attaching an intravascular device 234 to a vessel wall 254 of a body vessel 252 is illustrated. The attachment system 230 includes a tubular expandable body 232 and an intravascular device 234, similar to any of those hereinbefore described. The intravascular device 234 is disposed adjacent to the exterior side 240 of the tubular expandable body 232, and may be attached thereto by a biodegradable or non-biodegradable connection, such as those hereinbefore described. In the alternative, the intravascular device 234 may be detached from the tubular expandable body 232 and held in place within the body vessel 252 by the outward radial spring force of the tubular expandable 232, such that when deployed in a body vessel 252, the tubular expandable body 232 presses against the intravascular device 234. In such a configuration, the intravascular device 234 and the tubular expandable body 232 form a press fit against the vessel wall 254, with each of the tubular expandable body 232 and the intravascular device 234 contacting the vessel wall 254. In all other respects, the attachment system 230 may be similar to those hereinbefore described.

Another attachment system 330 for attaching an intravascular device 334 to a vessel wall 354 of a body vessel 352 is illustrated in FIG. 8. Like the attachment system 230 of FIG. 7, the attachment system 330 of FIG. 8 has an intravascular device 334 disposed adjacent to an exterior side 340 of a tubular expandable body 332. The attachment system 330 is similar in all respects to the attachment system 230 of FIG. 7, except that the tubular expandable body 332 has a kidney-bean shape, which may aid in anchoring the attachment system 330 against the vessel wall 354, as more surface area of the tubular expandable body 332 contacts the vessel wall 354 in this embodiment.

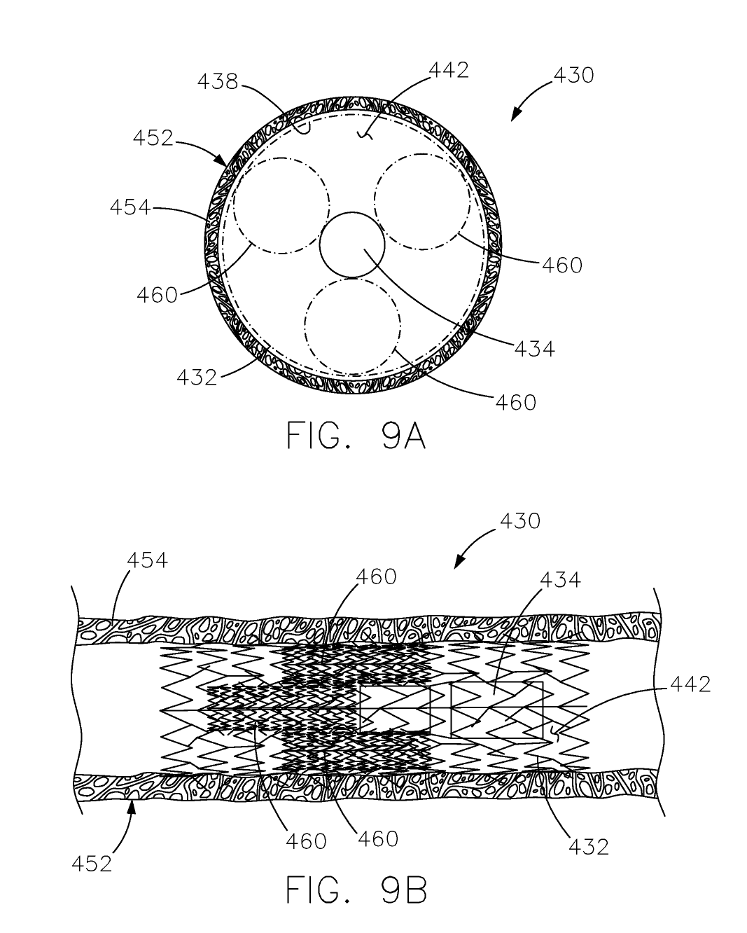

Now with reference to FIG. 9A, yet another attachment system 430 for attaching an intravascular device 434 to a vessel wall 454 of a body vessel 452 is illustrated. The attachment system 430 has a main tubular expandable body 432, which is configured to expand in the expanded state to contact the vessel wall 454 when deployed in the body vessel 452, similar to those tubular expandable bodies 32, 132, 232, 332 hereinbefore described. The main tubular expandable body 432 has an interior side 438 that defines a lumen 442 through the main tubular expandable body 432.

Three secondary tubular expandable bodies 460 are disposed adjacent to the interior side 438 of the main tubular expandable body 432 within the lumen 442 of the main tubular expandable body 432 to hold the intravascular device 434 within the lumen 442 of the main tubular expandable body 432. Each secondary tubular body 460 is similar to the main tubular expandable body 432, for example, having an expanded state and a collapsed state. It should be understood that although three secondary tubular expandable bodies 460 are shown in FIG. 9A, there could be any suitable number of secondary tubular expandable bodies 460.

In some embodiments, the secondary tubular expandable bodies 460 are attached to the main tubular expandable body 432 and/or the intravascular device 434, for example, with biodegradable or non-biodegradable connections, such as those hereinbefore described. The main and secondary tubular expandable bodies 432, 460 could be attached together by any suitable means, such as crimping, dimpling, twisting, adhesives, and hooks, by way of example.

In other embodiments, the secondary tubular expandable bodies 460 may be detached from the main tubular expandable body 432 and the intravascular device 434, the secondary tubular expandable bodies 460 holding the intravascular device 434 in place by means of a press fit, similar to that described with respect to FIG. 8. In other words, the secondary tubular expandable bodies 460 may exert a radial force against the main tubular expandable body 432 and the intravascular device 434 to hold the intravascular device 434 in place.

With reference to FIG. 9B, one way of deploying the attachment system 430 is illustrated. In this variation, the intravascular device 434 is detached from the secondary tubular expandable bodies 460. Further, a staged delivery of the tubular expandable bodies 432, 460 and the intravascular device 434 is contemplated. Thus, the tubular expandable bodies 432, 460 are first deployed into the body vessel 452. The main tubular expandable body 432 may be deployed prior to the secondary tubular expandable bodies 460, or they may be deployed together, for example, when the tubular expandable bodies 432, 460 are attached together. The secondary tubular expandable bodies 460 could form a barrier in the expanded state such that the intravascular device 434 is too large to migrate past the secondary tubular expandable bodies 460. Then, when the intravascular device 434 is deployed, it becomes wedged a lumen area between each of the secondary tubular expandable bodies 460 and is thereby held in place. Such a configuration may allow for the intravascular device 434 to be easily grasped for retrieval; or the intravascular device 434 may otherwise be easily removed, such as by retracting an attached retrieval member (not shown), which could be substantially similar to the retrieval members hereinbefore described (i.e., tether, cord, or drug-carrying channel).

As shown in FIG. 9B, the secondary tubular expandable bodies 460 need not be distributed in the common axial locations along the longitudinal axis. In FIG. 9B, one secondary tubular expandable body 460 is shown being located farther along the longitudinal axis than the other secondary tubular expandable bodies 460. In all other respects, the attachment system 430 may be similar to those hereinbefore described.

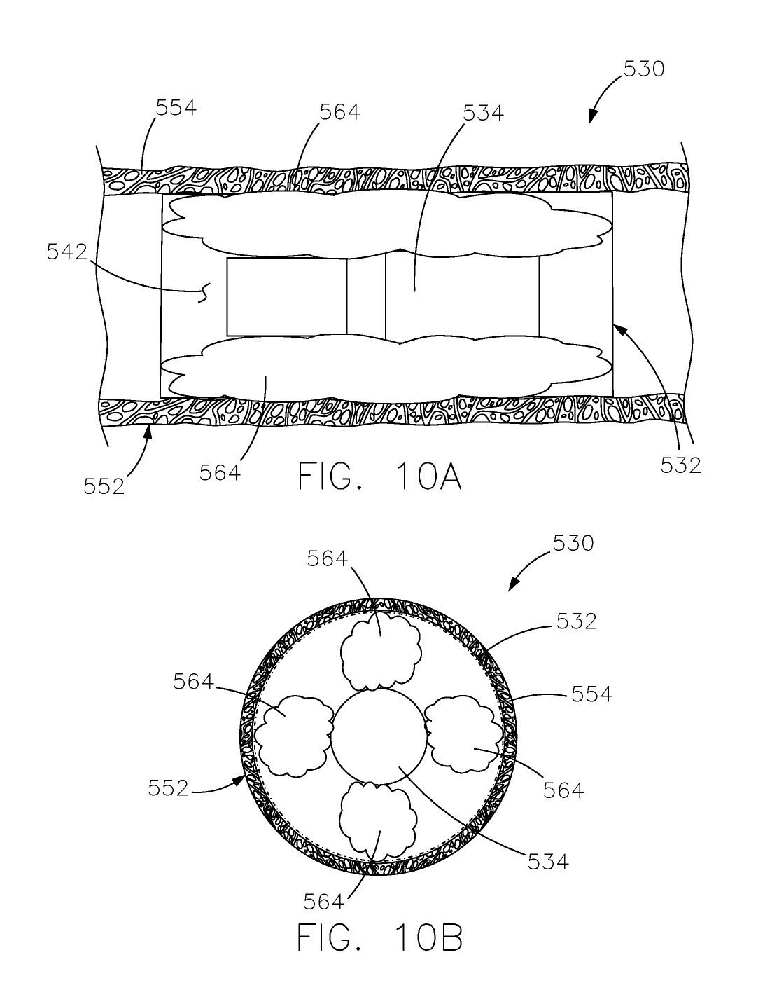

Now with reference to FIGS. 10A and 10B, yet another attachment system 530 for attaching an intravascular device 534 to a vessel wall 554 of a body vessel 552 is illustrated. A plurality of attachment members, such as balloons 564 in this embodiment, is configured to connect the intravascular device 534 within the lumen 542 of a tubular expandable body 532. Although only two balloons 564 are shown in FIG. 10A for clarity, it should be understood that four balloons 564, as illustrated in FIG. 10B, are present in this embodiment. It should also be understood that, in the alternative, any suitable number of balloons 564 could be used.

The balloons 564 are inflatable, having an inflated state and a deflated state. In some embodiments, the intravascular device 534 could be attached to the balloons 564, while in other embodiments, the intravascular device 534 may be detached from the balloons 564. The balloons 564 are preferably attached to the tubular expandable body 532, although this need not necessarily be true.

In FIGS. 10A and 10B, the tubular expandable body 532 is shown in an expanded state, similar to those tubular expandable bodies 32, 132, 232, 332, 432, 460 hereinbefore described. Thus, the tubular expandable body 532 contacts the vessel wall 554 along its diameter and its length. The balloons 564 are preferably initially deployed in a deflated state and then inflated to provide a tight fit around the intravascular device 534 to hold the same in place within the body vessel 552. As seen in FIG. 10B, the balloons 564 are staggered around the intravascular device 534, which allows fluid to flow around them in the body vessel 552.

The intravascular device 534 could be similar to any intravascular device described herein. In some embodiments, the balloons 564 may occupy a large amount of space in the lumen of the blood vessel 552, such that it is desirable to use an intravascular device having a hollow channel therethrough, such as the intravascular device 2934 described in FIG. 27B.

In another variation, in embodiments wherein flow is substantially blocked by the balloons 564, the system could be configured to detect a malfunction of the intravascular device 534 and deflate the balloons 564 to allow flow to travel through the blood vessel 552.

Similarly to the attachment system 430 of FIGS. 9A and 9B, the attachment system 530 of FIGS. 10A and 10B allows for a staged delivery of the attachment system 530. For example, the tubular expandable body 532 and the deflated balloons 564 could be introduced first into the body vessel 552, followed by the intravascular device 534. Then, the balloons 564 could be inflated to hold the intravascular device 534 in place. This configuration would allow the intravascular device 534 to be easily removed, as it could be easily grasped or pulled back into a catheter if the balloons 564 were deflated. In one variation, the balloons 564 could be made to slowly deflate over time, such that retrieval could be easily accomplished when the balloons 564 are slightly deflated. Such a configuration could help avoid the risk of migration that may exist with fully deflated balloons 564. In all other respects, it should be understood that the attachment system 530 could be substantially similar to those hereinbefore described.

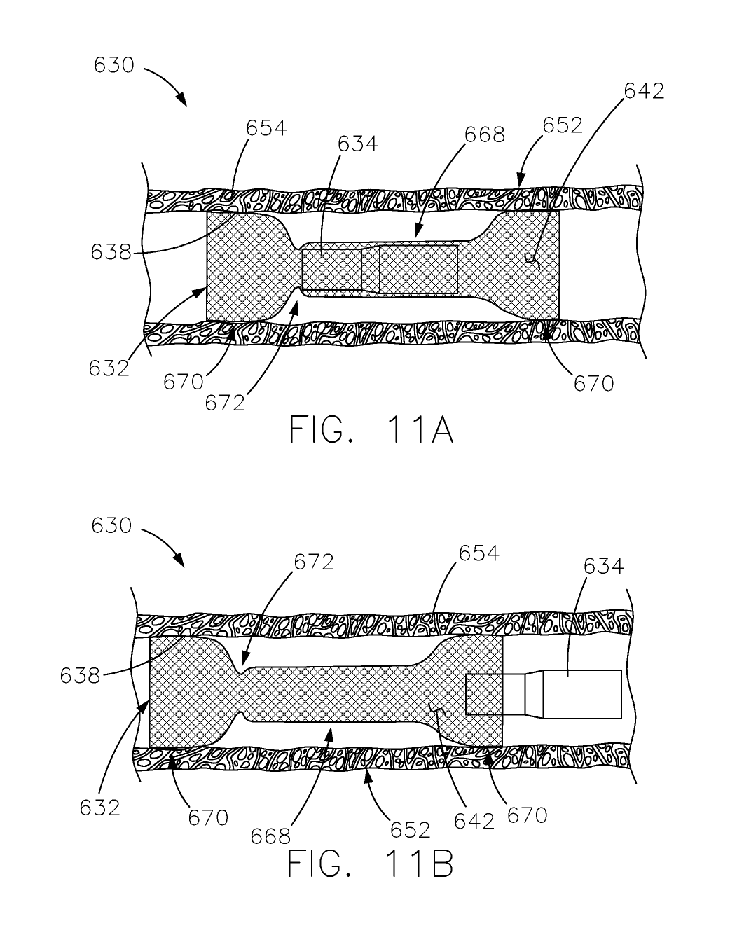

Yet another attachment system 630 for attaching an intravascular device 634 to a vessel wall 654 of a body vessel 652 embodying the principles of the present invention is illustrated in FIGS. 11A and 11 B. Similarly to the attachment systems 30, 130, 230, 330, 430, 530 hereinbefore described, the attachment system 630 of FIGS. 11A and 11 B includes an intravascular device 634 disposed within a lumen 642 of a tubular expandable body 632. The tubular expandable body 632 has an interior side 638 defining the lumen 642 through the tubular expandable body 632, and the tubular expandable body 632 is configured to move between an expanded state for attaching the intravascular device 634 within the body vessel 652 and a collapsed state for delivery and/or retrieval. Thus, the expanded diameter of the tubular expandable body 632 is larger than the collapsed diameter thereof. The tubular expandable body 632 is configured to contact the vessel wall 654 in the expanded state.

In this embodiment, the tubular expandable body 632 has a narrow portion 668 separating two wide portions 670. The narrow portion 668 has a diameter smaller than the diameter of the wide portions 668. The tubular expandable body 632 may also have an annular indentation 672 that has a diameter even smaller than that of the narrow portion 668. The intravascular device 634 is configured to be disposed within the lumen 642 of the tubular expandable body 632 and to have an outer diameter that is larger than at least the indentation 672. Thus, when deployed within the body vessel 652, the intravascular device 634 is trapped within the tubular expandable body 632 and cannot migrate past the indentation 672. In the alternative, the indentation 672 can be omitted, and the narrow portion 668 can have a diameter smaller than that of the intravascular device 634 to prevent the intravascular device 634 from migrating past the narrow portion 668 of the tubular expandable body 632.

This configuration, like some of the other attachment systems hereinbefore described, allows the attachment system 630 to be delivered via a staged delivery. For example, the tubular expandable 632 could be delivered prior to the delivery of the intravascular device 634. In all other respects, the attachment system 630 may be similar to those hereinbefore described.

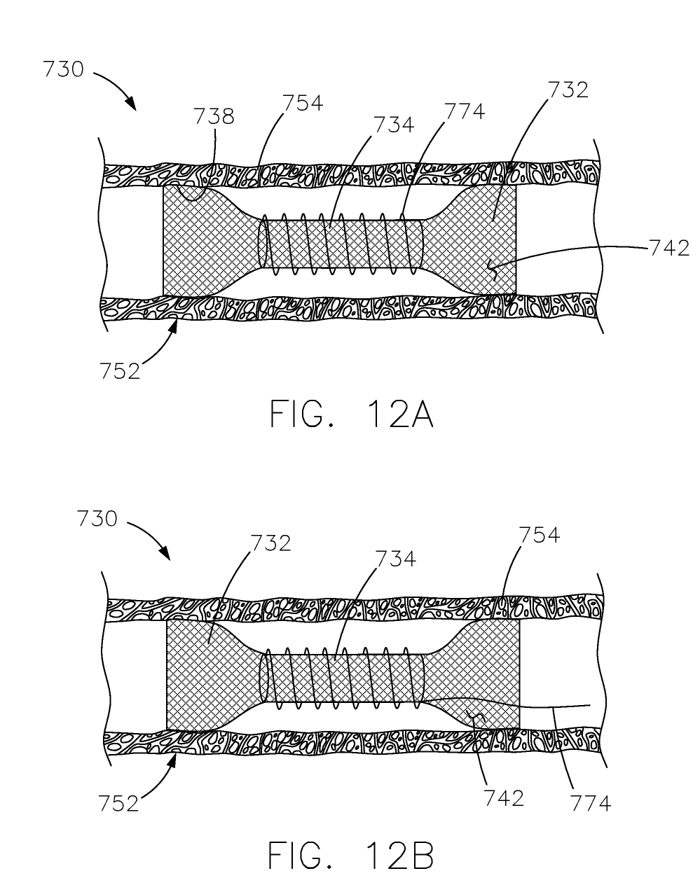

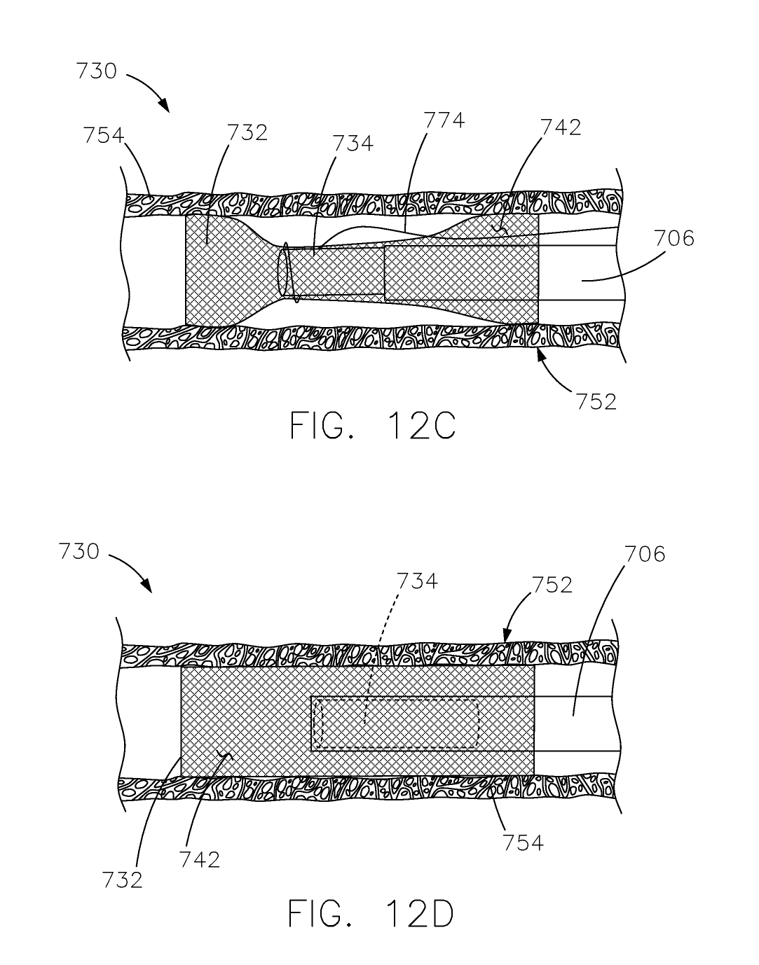

Now with reference to FIG. 12A, yet another attachment system 730 for attaching an intravascular device 734 to a vessel wall 754 of a body vessel 752 is illustrated. Like the attachment systems hereinbefore described, the attachment system 730 of FIG. 12A has a tubular expandable body 732, which has an interior side 738 defining a lumen 742 therethrough. The tubular expandable body 732 is configured to move between an expanded state and a collapsed state, the tubular expandable body 732 having a collapsed diameter in the collapsed state and an expanded diameter in the expanded state, wherein the expanded diameter is larger than the collapsed diameter. The tubular expandable body 732 is configured to contact the vessel wall 754 in the expanded stated when deployed in the body vessel 752.

A restricting member 774 is disposed around the tubular expandable body 732 to collapse a portion of the tubular expandable body 732. In this embodiment, the restricting member 774 is wrapped around the tubular expandable body 732 and the intravascular device 734 to attach the intravascular device 734 to the tubular expandable body 732. The restricting member 774 is attached around the tubular expandable body to center the intravascular device 734 within the lumen 742 of the tubular expandable body 732 along the longitudinal axis, thereby centering the intravascular device 734 within the body vessel 752, to form an hour glass shaped attachment system 730; however, the restricting member 774 could be wrapped around other portions of the tubular expandable body 732, without falling beyond the spirit and scope of the present invention. Furthermore, the intravascular device 734 need not be centered within the tubular expandable body 732, but rather, the intravascular device 734 could be attached to the tubular expandable body 734 off-center from the longitudinal axis and off-center from the center of the body vessel 752.

The restricting member 774 could be a marker band, a stitch, an SIS strand (described in further detail below), a fabric ring, a thread, a wire, a flexible tube, a portion of a cannula, or an elastic band, by way of example. Preferably, the restricting member 774 is a thread, wire, or band that may be wrapped around and unwrapped from the tubular expandable body 732.

With reference to FIG. 12B, the attachment system 730 is illustrated, wherein the restricting member 774 is slightly unraveled, or slightly unwrapped, from the tubular expandable body 732. With reference to FIG. 12C, the restricting member 774 is almost completely unwrapped, or unraveled, from the tubular expandable body 732. As the tubular expandable body 732 is unwrapped, it begins to expand at its middle portion, because the restricting member 774 is no longer providing a force on the unwrapped parts of the middle portion to collapse the tubular expandable body 774. As the tubular expandable body 732 expands, the intravascular device 734 begins to be released therefrom. A catheter 706 may be used to capture the intravascular device 734 as it is released from the tubular expandable body 732.

Now with reference to FIG. 12D, the attachment system 730 is illustrated, and the restricting member 774 has been fully removed from the tubular expandable body 732. As such, the tubular expandable body 732 has expanded completely to form a stent in the body vessel 752. The intravascular device 734 is thereby completely released form the tubular expandable body 732 and the restricting member 774, and it may be easily removed from the body vessel 752, for example, with the catheter 706. In all other respects, the attachment system 730 may be similar to those hereinbefore described.

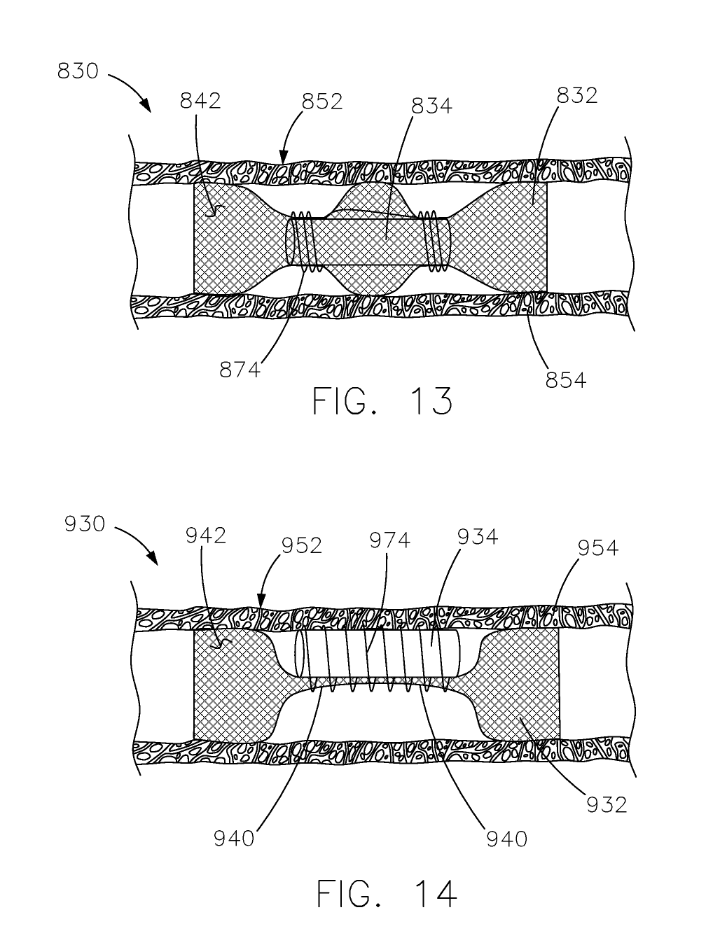

With reference to FIG. 13, another embodiment of an attachment system 830 for attaching an intravascular device 834 to a vessel wall 854 of a body vessel 852 is illustrated. The attachment system 830 of FIG. 13 is substantially similar to that of FIGS. 12A-12D, and as such, the attachment system 830 includes a tubular expandable body 832 configured to move between an expanded state and a collapsed state, an intravascular device 834 disposed within the lumen 842 of the tubular expandable body 832, and a restricting member 874 disposed around the tubular expandable body 832 to collapse a portion of the tubular expandable body 832. In this embodiment, the restricting member 874 may be wrapped around two separate portions of the tubular expandable body 832, or multiple restricting members 874 may be wrapped or otherwise disposed around separate portions of the tubular expandable body 832. In all other respects, the attachment system 830 may be similar to that of FIGS. 12A-12D, and to the other attachment systems described herein.

With reference to FIG. 14, yet another embodiment of an attachment system 930 for attaching an intravascular device 934 to a vessel wall 954 of a body vessel 952 is illustrated. The attachment system 930 of FIG. 14 is substantially similar to that of FIGS. 12A-12D and 13, and as such, the attachment system 930 includes a tubular expandable body 932 configured to move between an expanded state and a collapsed state, an intravascular device 934 attached to the tubular expandable body 932, and a restricting member 974 disposed around the tubular expandable body 932 to collapse a portion of the tubular expandable body 932. In this embodiment, the restricting member 974 is disposed adjacent to an exterior side 940 of the tubular expandable body 932 and is wrapped around the intravascular device 934 and the tubular expandable body 932 to attach the intravascular device 934 to the exterior side 940 of the tubular expandable body 932. The intravascular device 934 is attached outside the lumen 942 of the tubular expandable body 932 and off-center within the body vessel 952, or spaced apart from the longitudinal axis of the attachment system 930. The intravascular device 934 is located adjacent to the vessel wall 954. In some embodiments, the intravascular device 934 may be in contact with the vessel wall 954. In all other respects, the attachment system 930 may be similar to that of FIGS. 12A-12D and 13, and to the other attachment systems described herein.

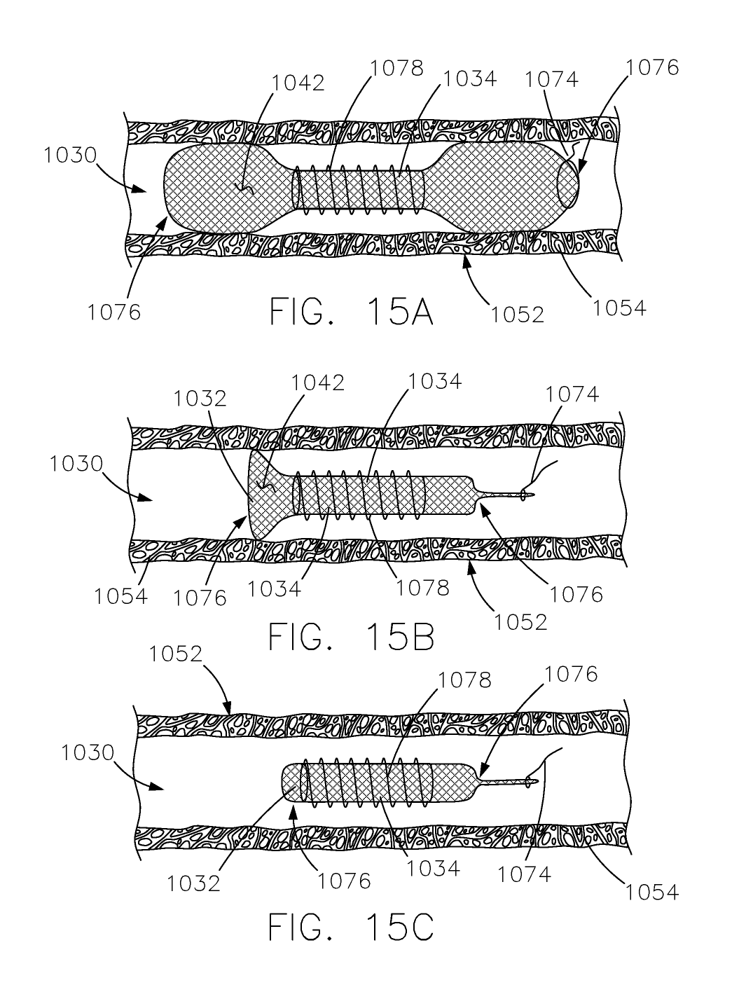

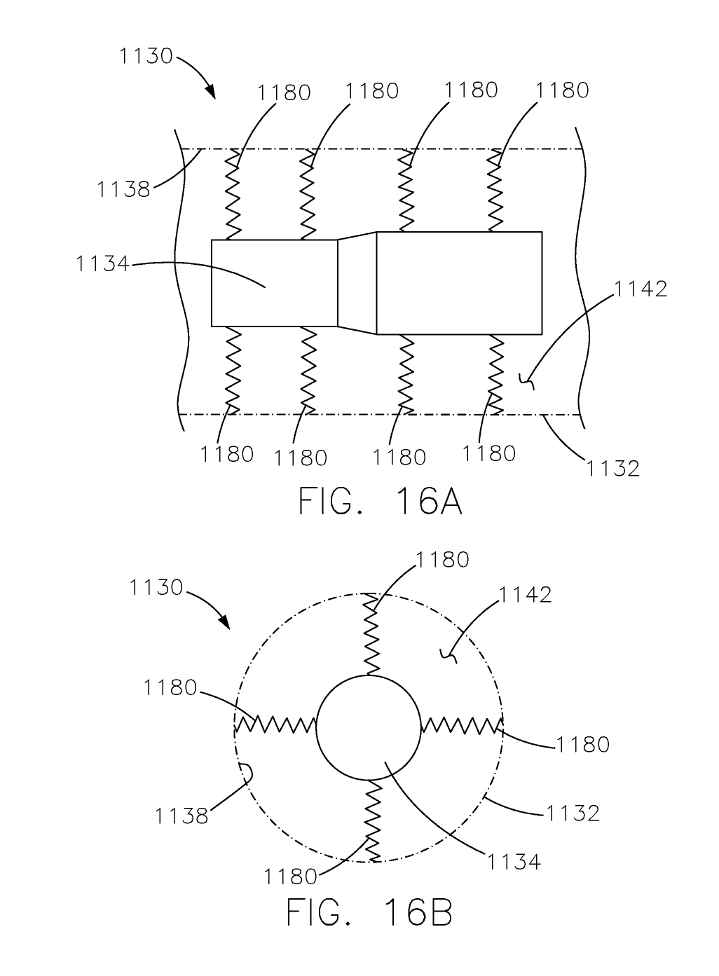

Now with reference to FIG. 15A, yet another attachment system 1030 for attaching an intravascular device 1034 to a vessel wall 1054 of a body vessel 1052 is illustrated. The attachment system 1030 includes a tubular expandable body 1032 configured to move between an expanded state and a collapsed state, an intravascular device 1034 disposed within the lumen 1042 of the tubular expandable body 1032, and a restricting member 1074 disposed around the tubular expandable body 1032 to collapse a portion of the tubular expandable body 1032. In this embodiment, the ends 1076 of the tubular expandable body 1032 have a tapering shape such that they are curved inward and away from the vessel wall 1054. The restricting member 1074 is threaded through an end 1076 of the tubular expandable body 1032; however, the restricting member 1074 may be attached to the tubular expandable body 1032 in any other suitable manner.

In this embodiment, the restricting member 1074 allows for retrieval of the entire attachment system 1030, including the tubular expandable body 1032 and the intravascular device 1034. With reference to FIG. 15B, the attachment system 1030 is illustrated in a partially collapsed state. As the restricting member 1074 is pulled, the tubular expandable body 1032 collapses into the collapsed state, the diameter of the tubular expandable body 1032 decreases, and the tubular expandable body 1032 is moved away from the vessel wall 1054. With reference to FIG. 15C, the entire attachment system 1030 may then be easily removed from the body vessel 1052, because the tubular expandable body 1032 is in the collapsed state.

In the attachment system 1030 of FIGS. 15A-15C, another restricting member 1078 may be disposed around the intravascular device 1034 and the tubular expandable body 1032, similarly to the restricting members 774, 874, 974 of FIGS. 12A-14, to collapse a middle portion of the tubular expandable body 1032 and attach the intravascular device 1034 to the tubular expandable body 1032. In the alternative, the tubular expandable body 1032 could have a narrow portion disposed around the intravascular device 1034 to hold the intravascular device 1034 in a desired position. It should be understood that, alternatively still, the intravascular device 1034 could be attached to the tubular expandable body 1032 in any other suitable manner.

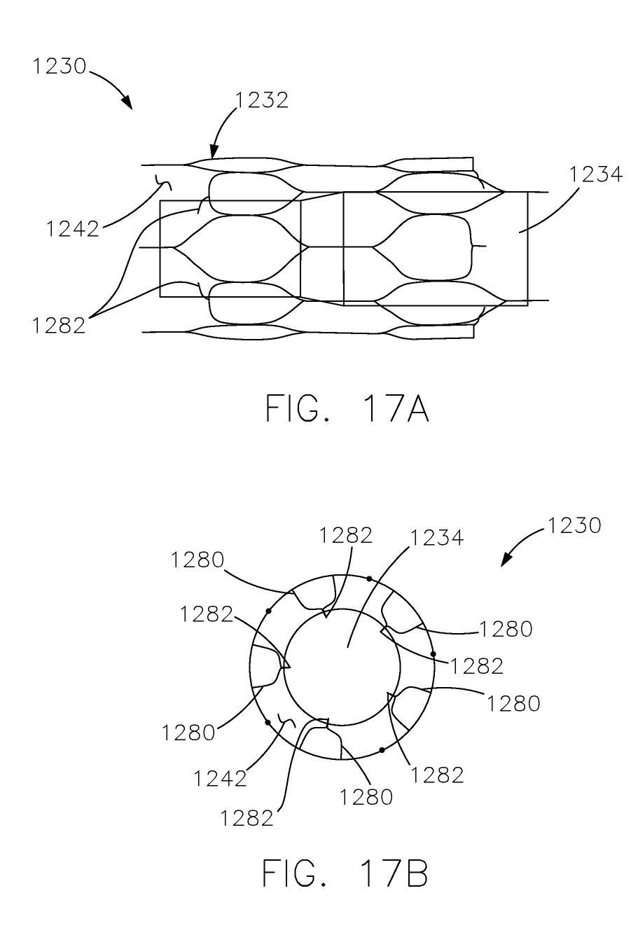

Referring now to FIGS. 16A and 16B, another attachment system 1130 for attaching an intravascular device 1134 to a vessel wall of a body vessel (not shown) is illustrated. Similarly to the previous attachment systems described herein, the attachment system 1130 of FIGS. 16A and 16B has a tubular expandable body 1132 having an interior side 1138 defining a lumen 1142 therethrough, and the tubular expandable body 1132 is configured to move between an expanded state and a collapsed state. For further description of the tubular expandable body 1132, please refer to the description of the tubular expandable body 32 of FIGS. 1A and 1B, which is herein incorporated by reference.

The intravascular device 1134 is disposed in the lumen 1142 of the tubular expandable body 1132. A plurality of attachment members 1180 extends into the lumen 1142 to connect the intravascular device 1134 to the tubular expandable body 1132. In this embodiment, the attachment members 1180 are springs having a polymeric coating disposed thereon, such that the intravascular device 1134 is suspended with a spring force and a dampening factor. As shown in FIGS. 9A and 9B, the attachment members 1180 hold the intravascular device 1134 away from the interior side 1138 of the tubular expandable body 1132 and therefore away from the vessel wall (not shown) to center the intravascular device 1134 within the lumen 1142 of the tubular expandable body 1132, although any other suitable configuration could be employed, such as an off-center configuration. Furthermore, this embodiment shows the attachment members 1180 extending around the radius of the intravascular device 1134 and along the length thereof, for added stability; however, it should be understood that a greater or fewer number of attachment members 1180 could be used, and they need not be placed symmetrically as illustrated herein. In all other respects, the attachment system 1130 may be similar to those hereinbefore described.

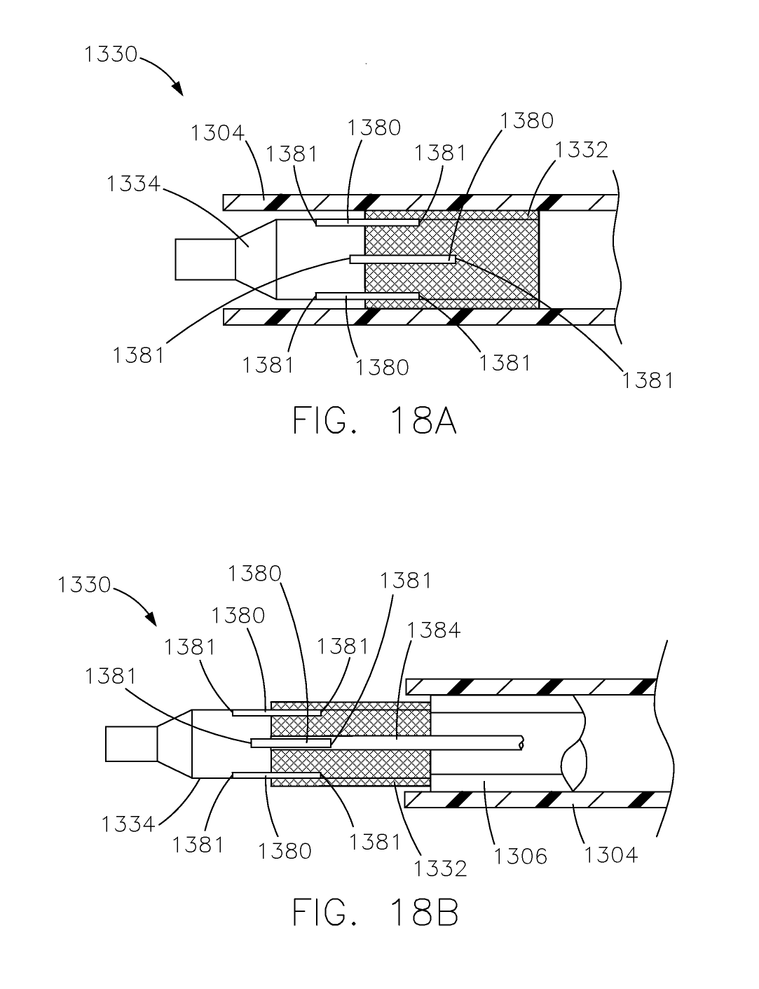

FIGS. 17A and 17B illustrate another attachment system 1230 for attaching an intravascular device 1234 to a vessel wall of a body vessel (not shown). Like the previous attachment systems described herein, the attachment system 1230 of FIGS. 17A and 17B has a tubular expandable body 1232, which is similar to those already described, and thus the details need not be repeated here. The intravascular device 1234 also may be similar to any of those hereinbefore described. The intravascular device 1234 is disposed within the lumen 1242 of the tubular expandable body 1232. In this embodiment, the tubular expandable body 1232 has a plurality of attachment members 1280 unitarily formed therewith. The attachment members 1280 extend into the lumen 1242 and attach the intravascular device 1234 to the tubular expandable body 1232, for example, using a biodegradable or non-biodegradable connection as described above. In the alternative, with reference to FIG. 17A, a portion 1282 of the attachment members 1280 may form a press fit with the intravascular device 1234 due to a radial force exerted on the intravascular device 1234 by the tubular expandable body 1232 and the vessel wall (not shown). Thus, the attachment members 1280 may connect the intravascular device 1234 to the tubular expandable body 1232 by a detached press fit, or by attaching the intravascular device 1234 to the tubular expandable body 1232.

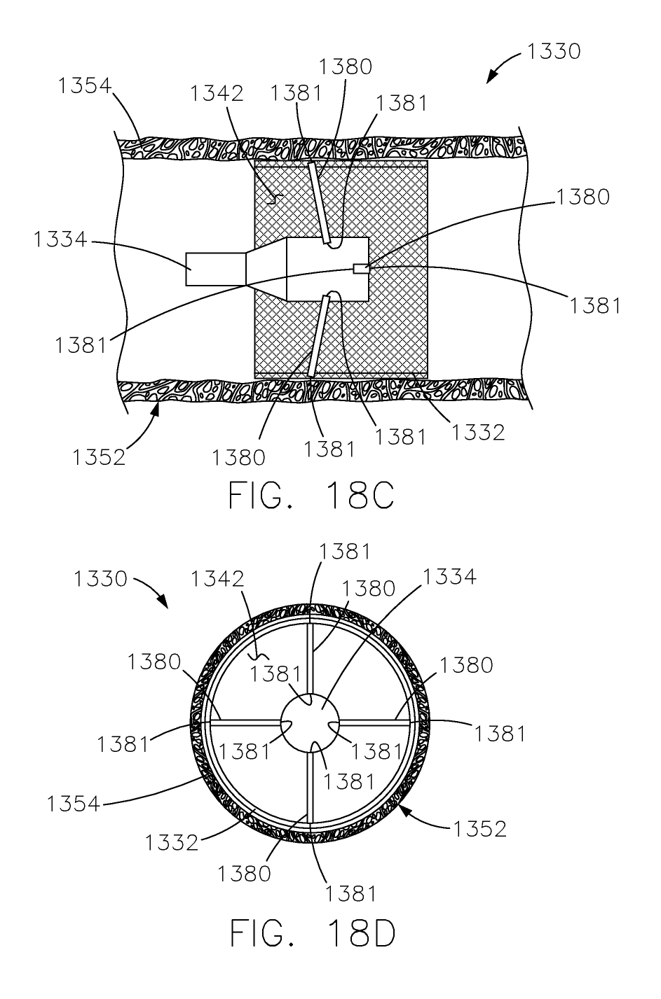

Now with reference to FIGS. 18A-18D, yet another attachment system 1330 for attaching an intravascular device 1334 to a vessel wall 1354 of a body vessel 1352 is illustrated. The attachment system 1330 includes a tubular expandable body 1332, which is similar to those hereinbefore described. A plurality of attachment members, such as struts 1380, connects the intravascular device 1334 to the tubular expandable body 1332. The struts 1380 are hingedly connected to the intravascular device 1334 and to the tubular expandable body 1332, at a plurality of pivot points 1381. Thus, when the tubular expandable body 1332 is in the collapsed state within a catheter sheath 1304 (see FIG. 18A), the struts 1380 lie parallel to the catheter sheath 1304 and the tubular expandable body 1332. It is contemplated that the struts 1380 may be formed of any suitable material, such as metal or polymer.

With reference to FIG. 18B, the attachment system 1330 is shown being deployed from the catheter sheath 1304. A pull rod 1384 may be removably or permanently attached to the intravascular device 1334. A catheter or cannula 1306 is configured to push the tubular expandable body 1332 from the catheter sheath 1304. The cannula 1306 has a hollow center to allow the pull rod 1384 to be pulled through the cannula 1306. To deploy the attachment system 1330 from the catheter sheath 1304, a user pushes the cannula 1306 to create a pushing force on the tubular expandable body 1332 and pulls the pull rod 1384 to create a pulling force on the intravascular device 1334. This deployment method helps ensure that the attachment system 1330 is properly placed within the body vessel 1352 because the tubular expandable body 1332 is forced to open upon deployment, when the tubular expandable body 1332 and the intravascular device 1334 are properly oriented. It should be understood that in some embodiments, the attachment system 1330 could alternatively be deployed by pushing the pull rod 1384 and pulling on the cannula 1306 or the tubular expandable body 1332.

With reference to FIGS. 18C and 18D, the attachment system 1330 is shown being deployed within the body vessel 1352 in the expanded state. When the tubular expandable body 1332 is in the expanded state, the struts 1380 are not parallel to the tubular expandable body 1332 or the vessel wall 1354. The struts 1380 suspend the intravascular device 1334 within the lumen 1342 of the tubular expandable body 1332. In this embodiment, the struts 1380 are configured to center the intravascular device 1334 within the lumen 1342. In some variations, the struts 1380 may become locked into a locked and open position when the tubular expandable body 1332 moves into the expanded state; in other variations, the locked position may be accomplished by virtue of a user manipulating the struts 1380 into the locked position through a combination of pushing the cannula 1306 and pulling the pull rod 1384.

Although four struts 1380 are shown surrounding a common axial portion of the intravascular device 1334, it should be understood that a greater or fewer number of struts 1380 could be attached to a given axial portion of the intravascular device 1334. Furthermore, the struts 1380 could be located at various axial locations along the longitudinal axis of the intravascular device 1334, wherein the longitudinal axis is defined as being parallel with the direction of fluid flow, which could give more stability to the intravascular device 1334.

The attachment system 1330 could be removed by collapsing the tubular expandable body 1332, using the pull rod 1384 and the cannula 1306. To collapse the tubular expandable body 1332, the pull rod 1384 and the cannula 1306 should be pushed or pulled in the opposite direction that each was pushed or pulled to deploy the attachment system 1330. Thus, in this embodiment, to remove the attachment system 1330, the pull rod 1384 should be pushed to push the intravascular device 1334 away from the tubular expandable body 1332. Such pushing causes the struts 1380 to pivot along the pivot points 1381 and collapse the tubular expandable body 1332. The tubular expandable body 1332 in most instances will cling to the vessel wall 1354, such that a pulling force need not be exerted on the tubular expandable body 1354 to collapse the tubular expandable body 1354, however, if desired, the tubular expandable body 1354 could be collapsed by pushing the pull rod 1384 and pulling on the tubular expandable body 1332.

FIG. 19 illustrates yet another attachment system 1430 for securing an intravascular device 1434 to a vessel wall of a body vessel (not shown). The attachment system 1430 includes the intravascular device 1434, which is similar to those intravascular devices hereinbefore described. The intravascular device 1434 has ends 1488 located along a length of the device 1434. A longitudinal axis L is defined along the length of the intravascular device 1434. In this embodiment, a plurality of arcuate struts 1486 is connected to the ends 1488 of the intravascular device 1434. Each strut 1486 has an attached end 1490 connected to one of the ends 1488 of the intravascular device 1432, and each strut 1486 has a free end 1492 for engaging the vessel wall of a body vessel (not shown) in an expanded state. The plurality of struts 1486 is configured to move along a strut path relative to the longitudinal axis between the expanded state for engaging the vessel wall and a collapsed state for delivery or retrieval of the attachment system 1430.

The attachment system 1430 may be held in a catheter sheath similar to those hereinbefore described, with the struts 1486 in the collapsed state, for inserting the attachment system 1430 into a body vessel. Upon deployment, the struts 1430 may open up to the expanded state to engage the vessel wall.

The struts 1486 may be formed of any suitable material, for example, a superelastic material, a nickel-based superalloy, stainless steel wire, cobalt-chromiumnickel-molybdenum-iron alloy, cobalt chrome-alloy, stress relieved metal (e.g., platinum), nickel-based superalloys, such as Inconel, or Nitinol, including linear elastic Nitinol and radiopaque Nitinol. The struts 1486 may preferably be formed of any appropriate material that will result in self-expanding struts 1486, wherein the attachment system 1430 is capable of being percutaneously inserted and deployed within a body cavity.

In one embodiment, the struts 1486 are made from Nitinol with a transition temperature that is slightly below normal body temperature of humans, which is about 98.6° F. Thus, when the attachment system 1430 is deployed in a body vessel and exposed to normal body temperature, the alloy of the struts 1486 will transform to austenite, that is, the remembered state, which for one embodiment of the present invention is the expanded state when the attachment system 1430 is deployed in the body vessel. To remove the attachment system 1430, the struts 1486 are cooled to transform the material to martensite which is more ductile than austenite, making the struts 1486 more malleable. As such, the struts 1486 can be more easily collapsed and pulled into a lumen of a catheter for removal.

In another embodiment, the struts 1486 are made from Nitinol with a transition temperature that is above normal body temperature of humans, which is about 98.6° F. Thus, when the attachment system 1430 is deployed in a body vessel and exposed to normal body temperature, the struts 1486 are in the martensitic state so that the struts 1486 are sufficiently ductile to bend or form into a desired shape, which for the present embodiment is the expanded state. To remove the attachment system 1430, the struts 1486 are heated to transform the alloy of the struts 1486 to austenite so that it becomes rigid and returns to a remembered state, which for the struts 1486 is a collapsed state.

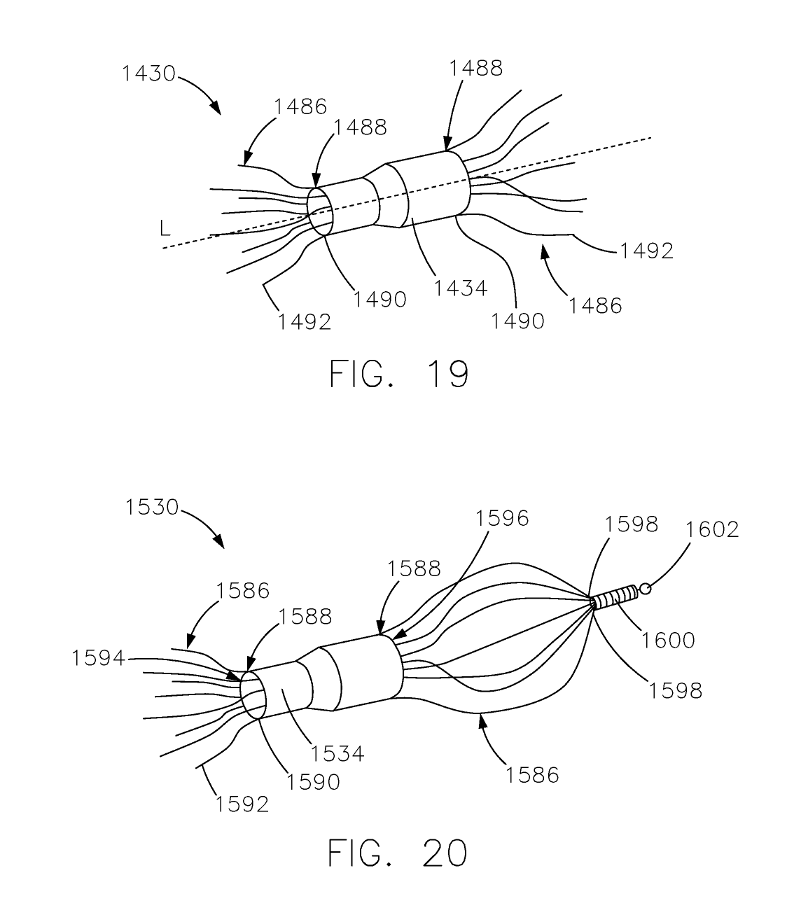

With reference to FIG. 20, yet another attachment system 1530 for attaching an intravascular device 1534 to a vessel wall of a body vessel is illustrated. The attachment system 1530 is similar to the attachment system 1430 of FIG. 19. As such, the attachment system 1530 includes the intravascular device 1534 and a plurality of struts 1586 attached to each end 1588 of the intravascular device 1534. On a distal side 1594 of the intravascular device 1534, each strut 1586 has an attached end 1590 attached to the intravascular device and a free end 1592 for engaging the vessel wall in the expanded state.

On a proximal side 1596 of the intravascular device 1534, the struts 1586 are attached to the intravascular device 1534 at attached ends 1590, similarly to the struts 1586 on the distal side 1594 of the intravascular device 1534. However, the struts 1586 on the proximal side 1596 do not have free ends; rather, the struts 1586 on the proximal side 1596 have proximal ends 1598 that are gathered together in a hub 1600. The hub 1600 may have a retrieval hook, such as an eyelet 1602, to aid in retrieval of the attachment system 1530. For example, to retrieve the attachment system 1530, a catheter may have a hook to grasp the eyelet 1602, which would allow the catheter to pull the proximal ends 1598 of the struts 1586, and thus the entire attachment system 1530, into a catheter sheath.

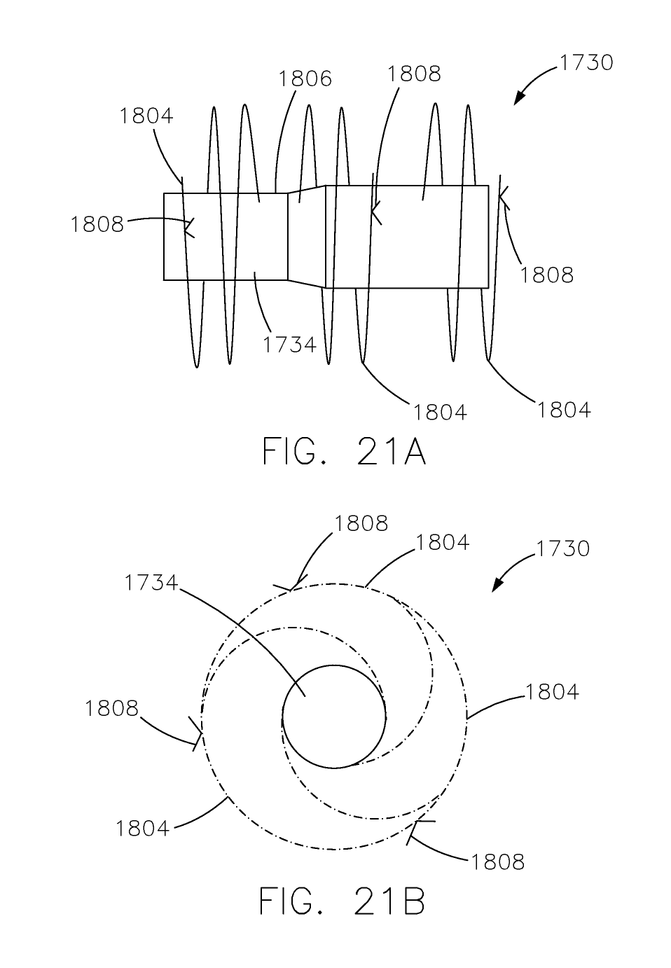

Now with reference to FIGS. 21A and 21 B, yet another attachment system 1730 for attaching an intravascular device 1734 to a vessel wall of a body vessel is illustrated. The attachment system 1730 includes the intravascular device 1734 and a plurality of coils 1804 attached to the exterior side 1806 of the intravascular device 1734. The coils 1804 may be attached in any suitable manner, such as by crimping, welding, press fit, wrapping the coil around the intravascular device 1734, or by the use of a lock and key. Each coil 1804 is configured to expand in an expanded state to engage the vessel wall, and each coil 1804 is further configured to collapse in a collapsed state for delivery or retrieval of the attachment system 1730. In one embodiment, the coils 1804 have a helical shape and extend from the intravascular device 1734 and outward to engage the vessel wall in the expanded state. The coils 1804 may each have barbs 1808 or other anchoring components for aiding in anchoring the attachment system 1730 to the vessel wall; however, it should be understood that the coils 1804 could anchor to the vessel wall through radial force alone.

Similarly to the struts 1486, 1586 of FIGS. 19 and 20, and the tubular expandable bodies 32, 132, 232, 332, 432, 532, 632, 732, 832, 932, 1032, 1132, 1232, 1332 hereinbefore described, the coils 1804 may be formed of any suitable material, for example, a superelastic material, a nickel-based superalloy, stainless steel wire, cobalt-chromiumnickel-molybdenum-iron alloy, cobalt chrome-alloy, stress relieved metal (e.g., platinum), nickel-based superalloys, such as Inconel, or Nitinol, including linear elastic Nitinol and radiopaque Nitinol. The coils 1804 may preferably be formed of any appropriate material that will result in self-expanding coils 1804, wherein the attachment system 1730 is capable of being percutaneously inserted and deployed within a body cavity.

In one embodiment, the coils 1804 are made from Nitinol with a transition temperature that is slightly below normal body temperature of humans, which is about 98.6° F. Thus, when the attachment system 1730 is deployed in a body vessel and exposed to normal body temperature, the alloy of the coils 1804 will transform to austenite, that is, the remembered state, which for one embodiment of the present invention is the expanded state when the attachment system 1730 is deployed in the body vessel. To remove the attachment system 1730, the coils 1804 are cooled to transform the material to martensite which is more ductile than austenite, making the coils 1804 more malleable. As such, the coils 1804 can be more easily collapsed and pulled into a lumen of a catheter for removal.

In another embodiment, the coils 1804 are made from Nitinol with a transition temperature that is above normal body temperature of humans, which is about 98.6° F. Thus, when the attachment system 1730 is deployed in a body vessel and exposed to normal body temperature, the coils 1804 are in the martensitic state so that the coils 1804 are sufficiently ductile to bend or form into a desired shape, which for the present embodiment is the expanded state. To remove the attachment system 1730, the coils 1804 are heated to transform the alloy of the coils 1804 to austenite so that it becomes rigid and returns to a remembered state, which for the coils 1804 is a collapsed state.

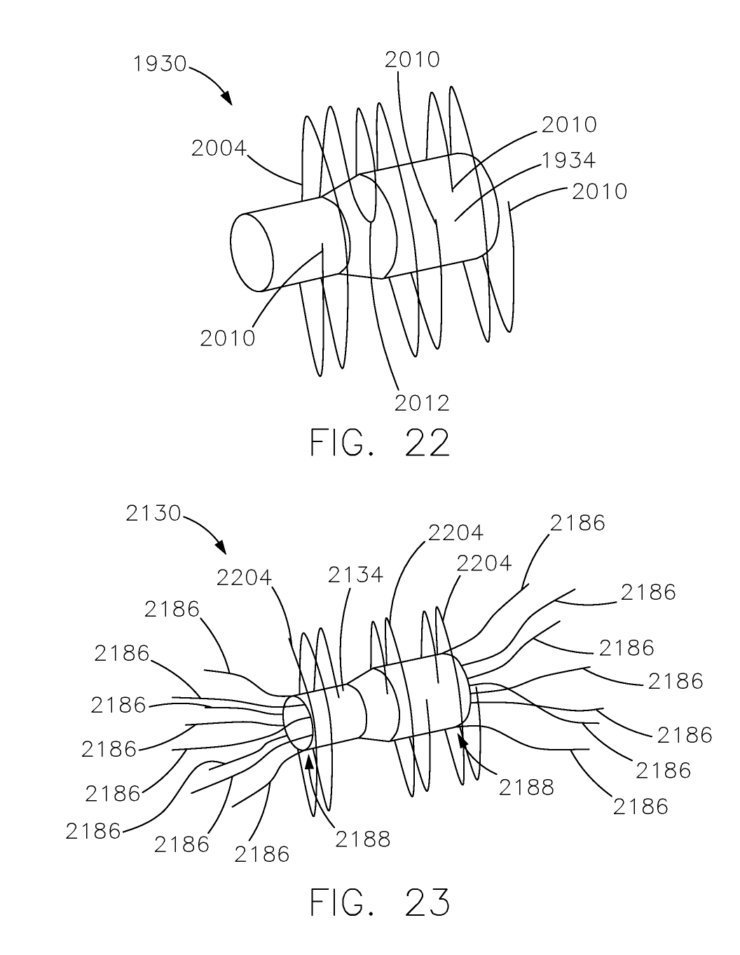

Now with reference to FIG. 22, another variation of an attachment system 1930 having coils 2004 is shown. The attachment system 1930 includes an intravascular device 1934 with a plurality of coils 2004 attached thereto. The coils 2004 extend outwardly from the intravascular device 1934 in an expanded state. The coils 2004 may be made of the same material as the coils 1804 of FIGS. 21A and 21 B, and like the coils 1804 of FIGS. 21A and 21B, the coils 2004 are configured to move between an expanded state and a collapsed state.

In this embodiment, the coils 2004 each have a free end 2010 that is configured to refrain from contacting the vessel wall in the expanded state. One way of accomplishing this arrangement is illustrated in FIG. 22, wherein the coils 2004 change direction at an outer point 2012, and thus the free end 2010 is located inwardly from the outer point 2012 and away from the vessel wall. This configuration may be effective to reduce trauma on the vessel wall.

Now with reference to FIG. 23, yet another attachment system 2130 for attaching an intravascular device 2134 to a vessel wall is illustrated. Similarly to the attachment system 1730 of FIGS. 21A and 21B, the attachment system 2130 of FIG. 23 includes an intravascular device 2134 and a plurality of coils 2204 attached thereto and extending outward in a helical pattern from the intravascular device 2134 toward a vessel wall in the expanded state when deployed in a body vessel. The attachment system 2130 may be similar to the attachment system 1730 of FIGS. 21A and 21B, except that the attachment system 2130 also includes a plurality of struts 2186 attached to each end 2188 of the intravascular device 2134. The plurality of struts 2186 may be similar to the plurality of struts 1486 of FIG. 19, which is described above. In all other respects, the attachment system 2130 may be similar to those hereinbefore described.

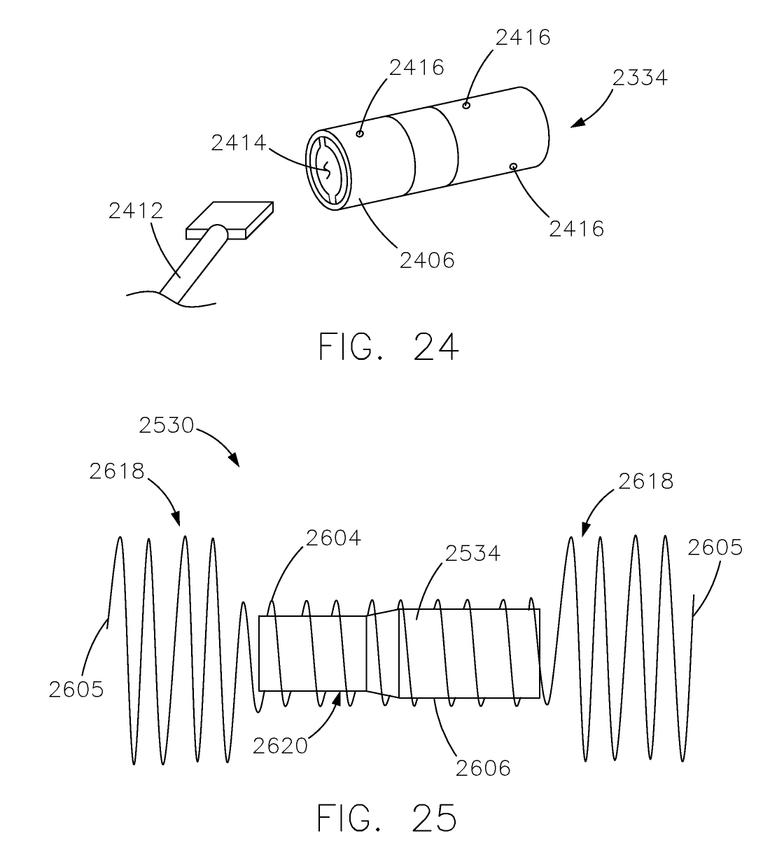

Turning now to FIG. 24, one variation of an intravascular device 2334 that may be used with any of the coils 1804, 2004, 2204 of FIGS. 21A-23 is illustrated. The intravascular device 2334 may be similar to those hereinbefore, described, and in addition, may have a plurality of connecting features to releasably retaining the coils 1804, 2004, 2204 to the intravascular device 2334. In other words, the coils 1804, 2004, 2204 may be removably attached to the intravascular device. In this embodiment, the coils 1804, 2004, 2204 may be released intravascularly from the intravascular device 2334 through the use of a key 2412. The key 2412 may be inserted into an opening 2414 within the intravascular device 2334 and turned to release a holding force that is being applied to the coils 1804, 2004, 2204. Upon releasing the force that holds the coils 1804, 2004, 2204 in place, the coils 1804, 2004, 2204 may be released from the intravascular device 2334. In this case, the coils 1804, 2004, 2204 may be held within apertures 2416 located along the exterior side 2406 of the intravascular device 2334 and released from the apertures 2416 upon insertion of the key 2412 into the opening 2414 of the intravascular device 2334 and turning the key 2412 to unlock and release the coils 1804, 2004, 2204. In some embodiments, the opening 2414 or the area surrounding it and the key 2412 may be radiopaque to assist with locating the key 2412 within the opening 2414 to release the coils 1804, 2004, 2204.

Yet another attachment system 2530 for attaching an intravascular device 2534 to a vessel wall is illustrated in FIG. 25. The attachment system 2530 includes the intravascular device 2534, which may be similar to those hereinbefore described. Further, the attachment system 2530 includes a coil 2604 attached to the intravascular device 2534. The coil 2604 has a dumbbell shape, wherein a pair of end sections 2618 surrounds a middle section 2620. Furthermore, in the expanded state, each end section 2618 has a diameter larger than the diameter of the middle section 2620, and the diameter of each of the end sections 2618 is about equal. Preferably, the middle section 2620 is wrapped around the exterior side 2606 of the intravascular device 2534 to attach the coil 2604 to the intravascular device 2534. It should be understood, however, that the coil 2604 could alternatively be connected to the intravascular device 2534 in other ways, such as through the use of adhesive, or by crimping, or by any other suitable method.

The attachment system 2530 may be retrieved, among other ways, by grasping an end 2605 of the coil 2604 and pulling the end 2605 into a cannula sheath, to pull one of the end sections 2618 away from the vessel wall 2554. Then, the cannula sheath could be moved over the middle section 2620 and the opposite end section 2618 to remove the entire system 2530 from the vessel 2552.

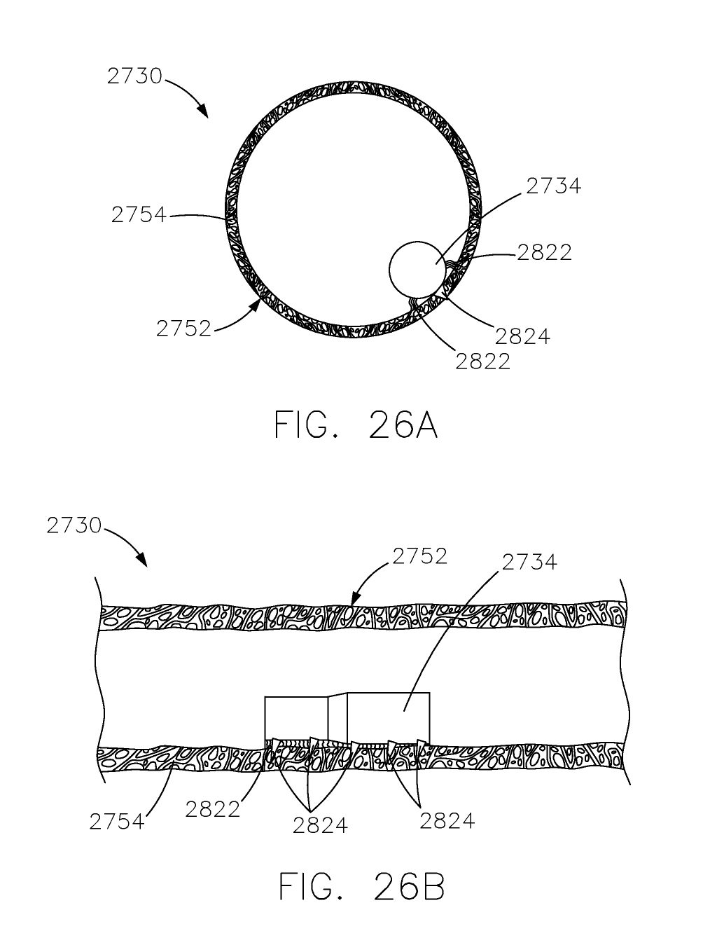

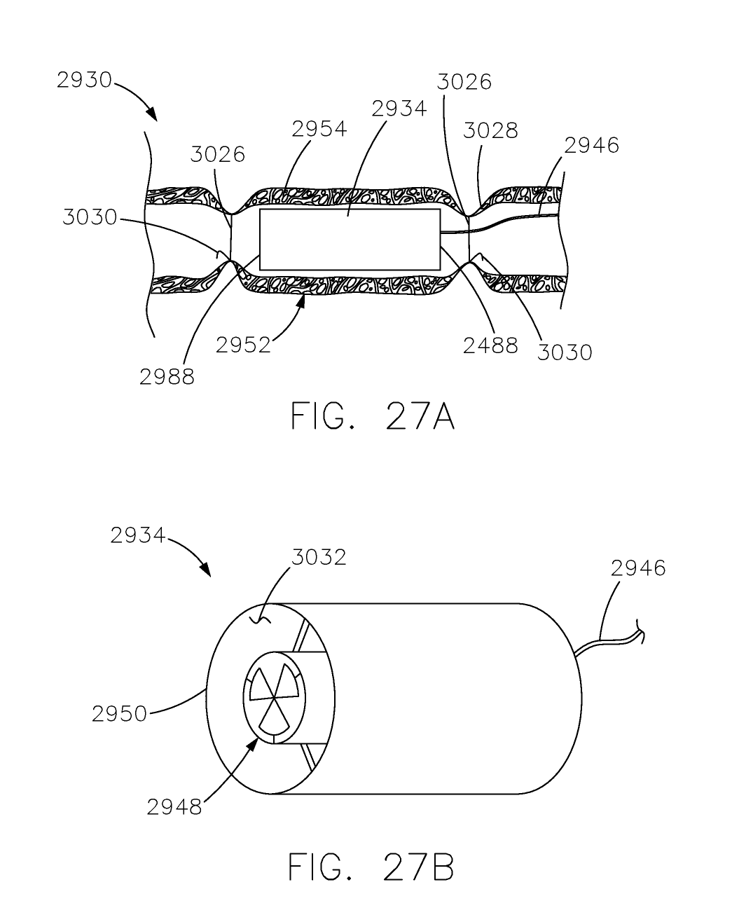

Now with reference to FIGS. 26A and 26B, another attachment system 2730 for attaching an intravascular device 2734 to a vessel wall 2754 of a body vessel 2752 is illustrated. The attachment system 2730 includes the intravascular device 2734 and biological attachment material 2822 connected to the intravascular device 2734. Furthermore, the attachment system 2730 may include a plurality of attachment members, such as hooks or barbs 2824, to further aid in anchoring the intravascular device 2734 to the vessel wall 2754. The intravascular device 2734 may be similar to those hereinbefore described.

The biological attachment material 2822 is configured to attach the intravascular device 2734 to the vessel wall 2754. The biological attachment material 2822 may comprise an extracellular matrix (ECM). As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In one particular embodiment, the extracellular matrix is comprised of small intestinal submucosa (SIS). As known, SIS is a resorbable, acellular, naturally occurring tissue matrix composed of extracellular matrix (ECM) proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS may be used to induce site-specific remodeling of both organs and tissues depending on the site of implantation. In practice, host cells are stimulated to proliferate and differentiate into site-specific connective tissue structures, which have been shown to completely replace the SIS material in time.

In this embodiment, SIS is attached to the intravascular device 2734 to assist with attaching the intravascular device 2734 to the wall 2754 of a body vessel 2752. The SIS adheres to the wall 2754 of the body vessel 2752 and promotes body tissue growth within the body vessel 2752. SIS has a natural adherence or wetability to body fluids and connective cells comprising the connective tissue of the walls of a body vessel. If the attachment system 2730 is intended to be permanently implanted within the body vessel 2752, the attachment system 2730 is positioned such that the host cells of the wall will adhere to the SIS and subsequently differentiate, growing into the SIS and eventually forming a bond of body tissue to the intravascular device 2734. In another particular embodiment, the SIS may be used to temporarily adhere the intravascular device 2734 to the wall 2754 of the body vessel 2752. If the intravascular device 2734 is only deployed within the body vessel 2752 temporarily, host cells of the vessel wall 2754 may adhere to the intravascular device 2734, but will not differentiate, allowing for later retrieval of the intravascular device 2734 from the body vessel 2752.

Referring now to FIG. 27A, yet another attachment system 2930 for securing an intravascular device 2934 within a body vessel 2952 is illustrated. The attachment system 2930 includes the intravascular device 2934 and a pair of entrapment devices located adjacent to each end 2988 of the intravascular device 2934. In this embodiment, the entrapment devices are a pair of sutures 3026. The sutures 3026 are configured to attach to the vessel wall 2954 as shown and prevent the intravascular device 2934 from migrating within the body vessel 2952 past either of the sutures 3026.

The sutures 3026 may be sewn into an exterior side 3028 of the body vessel 2952 or wrapped around the exterior side 3028 of the body vessel 2952 to partially collapse the body vessel 2952 at locations surrounding the ends 2988 of the intravascular device 2934. The sutures 3026 collapse the body vessel 2952 to a diameter smaller than the diameter of the intravascular device 2934. In this way, the intravascular device 2934 is prevented from migrating past either of the sutures 3026. Each suture 3026 may be degradable over time to help facilitate removal of the intravascular device 2934, if desired. In addition, or in the alternative, the sutures 3026 could have release hooks 3030 for releasing the sutures 3026 and retrieving the intravascular device 2934.

The attachment device 2930 of FIG. 27A is amenable to staged deployment of the attachment system 2930. For example, one suture 3026 could be attached to the body vessel 2952 first, followed by deployment of the intravascular device 2934, followed by attachment of the second suture 3026 to trap the intravascular device 2934 between the pair of sutures 3026. The system 2930 could also be implanted in any other suitable manner, without falling beyond the spirit and scope of the present invention.

Now with reference to FIG. 27B, the intravascular device 2934 of FIG. 27A is shown as a cardiac assist device having a pump 2948 surrounded by a pump housing 2950 and having a tether 2946 for providing power to rotate the pump 2948; however, it should be understood that the intravascular device 2934 could be any functional intravascular device 2934, as hereinbefore described. The intravascular device 2934 has portions forming a hollow section 3032 configured to allow fluid to flow therethrough in a substantially unimpeded manner. Such a hollow section 3032 may be advantageous for cardiac assist devices that are not intended to completely replace the flow rate of the human heart, and thus, it is desired to allow fluid to flow around the pump 2948 as well as through the pump 2948.

Although the hollow section 3032 is shown as being used with the embodiment of FIGS. 27A and 27B, it should be understood that the hollow section 3032 may be used with any other of the attachment systems described herein, where it may be desirable to allow more fluid to flow around the intravascular device.

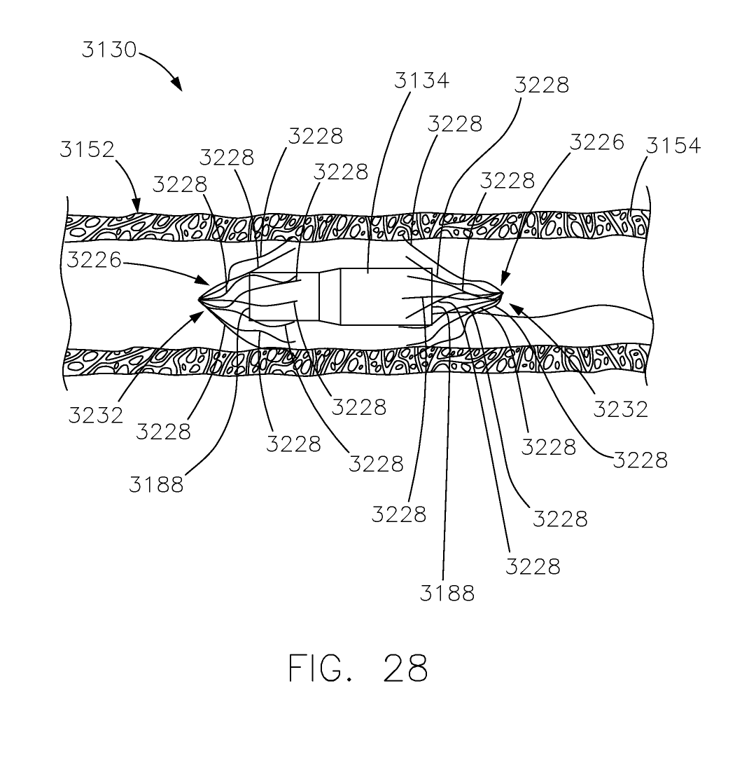

Now with reference to FIG. 28, another attachment system 3130 for securing an intravascular device 3134 within a body vessel 3152 is illustrated. Similarly to the embodiment of FIG. 27A, the attachment system 3130 illustrated in FIG. 28 includes the intravascular device 3134, which may be similar to any intravascular device hereinbefore described, and a pair of entrapment devices located adjacent to each end 3188 of the intravascular device 3134. In this embodiment, the entrapment devices are a pair of filters 3226. The filters 3226 are configured to attach to the vessel wall 3154 as shown and prevent the intravascular device 3134 from migrating within the body vessel 3152 past either of the filters 3226.

More particularly, each filter 3226 comprises a plurality of struts 3228. The struts 3228 of each filter 3226 form a filter basket, and each strut 3228 is configured to anchor the filter 3226 to the vessel wall 3154. As such, the filters 3226 are configured to be anchored to the vessel wall 3154 adjacent each end 3188 of the intravascular device to entrap the intravascular device 3134 therebetween. The struts 3228 may optionally have anchoring members, such as hooks or barbs (not shown) located on the free ends 3230 of each struts 3228. Each filter 3226 has a collapsed state for delivery and retrieval and an expanded state for engaging the vessel wall 3154. Each strut 3228 in the expanded state extends from an attached end 3232 to a free end 3230, each strut 3228 extending arcuately from the attached end 3232 to the free end 3230; however, it should be understood that the struts 3228 could have other configurations, such as a straight, non-arcuate configuration.

Similarly to the struts 1486, 1586 of FIGS. 19 and 20, the coils 1804, 2004, 2204, 2604 of FIGS. 21A-23 and 25, and the tubular expandable bodies 32, 132, 232, 332, 432, 532, 632, 732, 832, 932, 1032, 1132, 1232, 1332 hereinbefore described, the struts 3228 of each filter 3226 may be formed of any suitable material, for example, a superelastic material, a nickel-based superalloy, stainless steel wire, cobalt-chromium-nickelmolybdenum-iron alloy, cobalt chrome-alloy, stress relieved metal (e.g., platinum), nickel-based superalloys, such as Inconel, or Nitinol, including linear elastic Nitinol and radiopaque Nitinol. The struts 3228 of each filter 3226 may preferably be formed of any appropriate material that will result in self-expanding struts 3228, wherein the filters 3226 are capable of being percutaneously inserted and deployed within a body cavity.

In one embodiment, the struts 3228 of the filters 3226 are made from Nitinol with a transition temperature that is slightly below normal body temperature of humans, which is about 98.6° F. Thus, when the filters 3226 are deployed in a body vessel and exposed to normal body temperature, the alloy of the struts 3228 will transform to austenite, that is, the remembered state, which for one embodiment of the present invention is the expanded state when the filters 3226 are deployed in the body vessel 3152. To remove the filters 3226, the struts 3228 are cooled to transform the material to martensite which is more ductile than austenite, making the struts 3228 more malleable. As such, the struts 3228 can be more easily collapsed and pulled into a lumen of a catheter for removal.

In another embodiment, the struts 3228 of the filters 3226 are made from Nitinol with a transition temperature that is above normal body temperature of humans, which is about 98.6° F. Thus, when the attachment system 3130 is deployed in a body vessel and exposed to normal body temperature, the struts 3228 of the filters 3226 are in the martensitic state so that the struts 3228 are sufficiently ductile to bend or form into a desired shape, which for the present embodiment is the expanded state. To remove the filters 3226, the struts 3228 are heated to transform the alloy of the struts 3228 to austenite so that it becomes rigid and returns to a remembered state, which for the filters 3226 is a collapsed state.

In some embodiments, the filters 3226 may have a hub surrounding the attached ends 3232 and a retrieval hook extending from the hub (not shown) to aid in retrieval of the filters 3226, which may be similar to the hub 1600 and retrieval hook discussed above with respect to FIG. 20. The discussion above of the hub 1600 and retrieval hook of FIG. 20 is herein incorporated by reference.

The attachment device 3130 of FIG. 28 is amenable to staged deployment of the attachment system 3130. For example, one filter 3226 could be deployed within the body vessel 2952 first, for example, with the use of a delivery system similar to the delivery system 100 described with respect to FIGS. 2A-3. Such deployment the first filter 3226 may be followed by deployment of the intravascular device 3134, which may be followed by deployment of the second filter 3226 to trap the intravascular device 3134 between the pair of filters 3226. The system 3130 could also be implanted in any other suitable manner, without falling beyond the spirit and scope of the present invention.

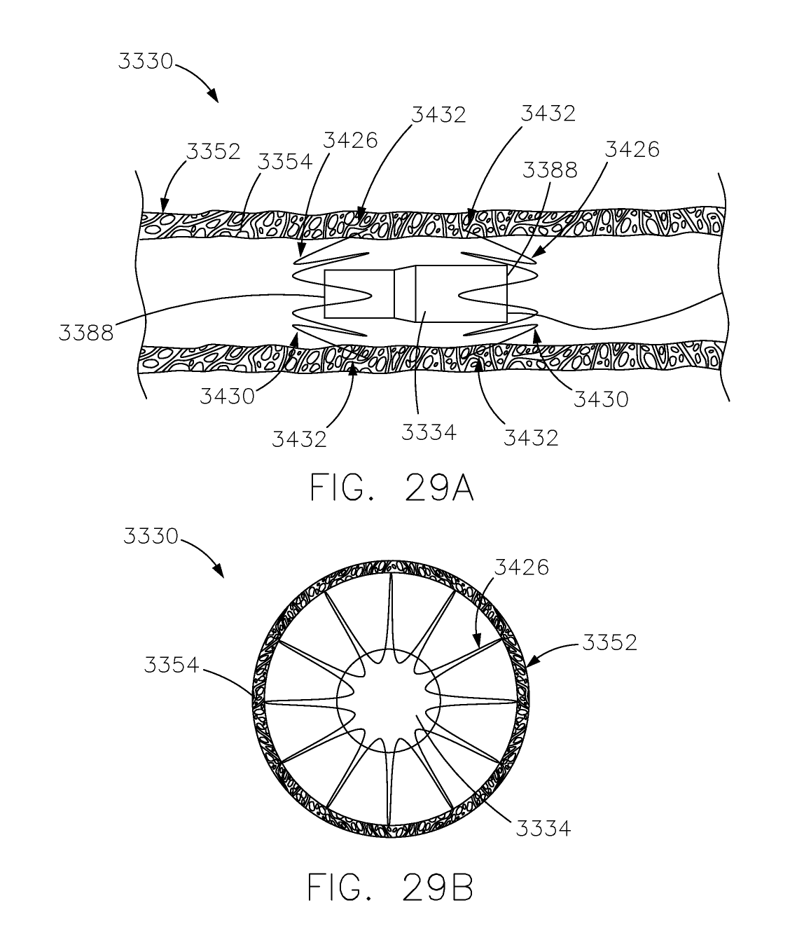

Now with reference to FIGS. 29A and 29B, yet another attachment system 3330 for securing an intravascular device 3334 within a body vessel 3352 is illustrated. Similarly to the embodiments of FIGS. 27A and 28, the attachment system 3330 illustrated in FIGS. 29A and 29B includes the intravascular device 3334, which may be similar to any intravascular device hereinbefore described, and a pair of entrapment devices located adjacent to each end 3388 of the intravascular device 3334. In this embodiment, the entrapment devices are a pair of tubular expandable bodies 3426. The tubular expandable bodies 3426 are configured to attach to the vessel wall 3354 as shown and prevent the intravascular device 3334 from migrating within the body vessel 3352 past either of the tubular expandable bodies 3426.

Like the attachment systems 2930, 3130 of FIGS. 27A and 28, the tubular expandable bodies 3426 of the attachment system 3330 of FIGS. 29A and 29B are configured to be detached from the intravascular device 3334 and to merely trap the intravascular device 3334 between the tubular expandable bodies 3426. The tubular expandable bodies 3426 may be similar to the tubular expandable bodies hereinbefore described, except that the tubular expandable bodies 3426 should taper from one side to another, such that a first end 3430 of each tubular expandable body 3426 has a diameter smaller than the diameter of the intravascular device 3334 and a second end 3432 has a diameter large enough to contact the vessel wall 3354 to anchor the tubular expandable body 3426 to the vessel wall 3354. Anchoring members (not shown), such as barbs or hooks, may be included on the second end 3432 of each tubular expandable body 3426 to aid in anchoring each tubular expandable body 3426 to the vessel wall 3354.

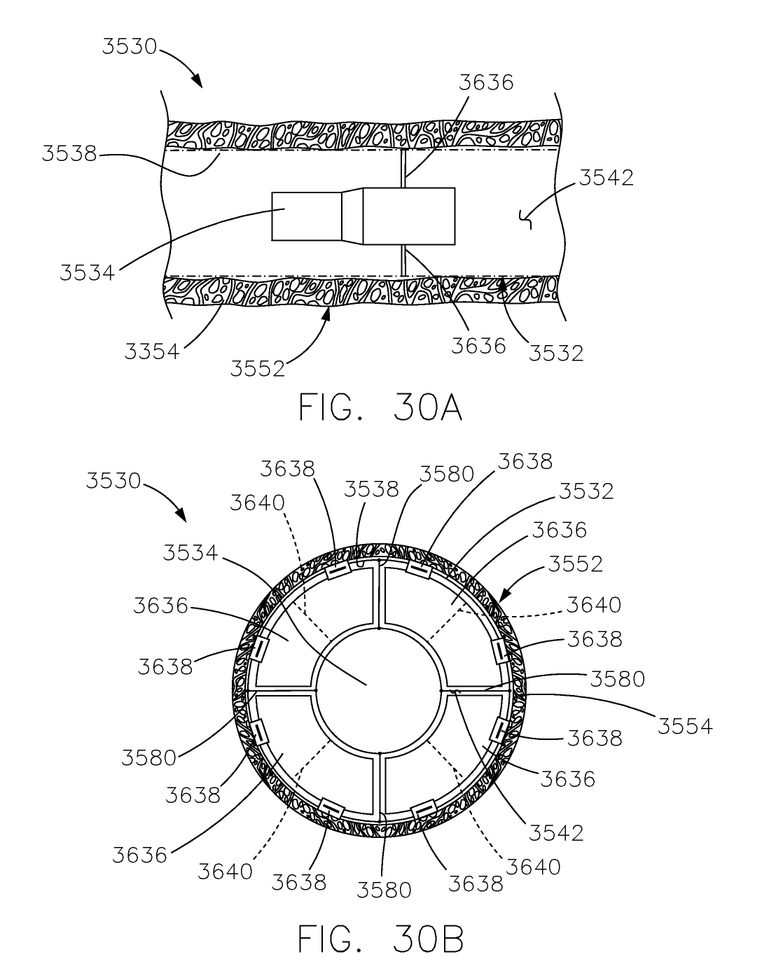

Now referring to FIGS. 30A and 30B, another attachment system 3530 for attaching an intravascular device 3534 to a vessel wall 3554 of a body vessel 3552 is illustrated. The attachment system 3530 includes a tubular expandable body 3532, which is similar to those hereinbefore described, particularly with respect to FIGS. 1A-5, 10A, 10B, 16A, 16B, and 18A-18D. As such, the tubular expandable body 3532 has an interior side 3538 defining a lumen 3542 within the tubular expandable body 3532 and is configured to move between a collapsed state and an expanded state. A plurality of attachment members, such as struts 3580, extends from the interior side 3538 of the tubular expandable body 3532 into the lumen 3542 thereof to connect the intravascular device 3534 to the tubular expandable body 3532. Thus, the intravascular device 3532 is located within the lumen 3542 of the tubular expandable body 3532. The struts 3580 are connected to the intravascular device 3534 and to the tubular expandable body 3532 in any suitable manner, such as any of the ways hereinbefore described, and the struts 3580 may have any suitable configuration, such as any of those configurations hereinbefore described. In this embodiment, the tubular expandable body 3532 is configured to attach to the vessel wall 3554 along its exterior side 3540, for example, through radial force and/or through the use of attachment members, such as barbs or hooks (not shown).

In some applications, it may be desirable to prevent fluid from flowing in a reverse direction, as compared to a main axial flow direction, or to keep flow momentum moving ante-grade, rather than retro-grade. For example, when the intravascular device 3534 is a pump, it may be desirable to ensure that fluid does not flow from the outlet end of the pump and back around the pump into the inlet end, or it may be desirable to at least decrease such flow. In other words, it may be desirable to stop or lessen retro-grade flow around a concentric motor; instead, it may be desirable to ensure that fluid only flows downstream, which would result in a more efficient pump. Thus, the present embodiment comprises a plurality of one-way valves 3636 disposed around the intravascular device 3534 when the tubular expandable body 3532 is in the expanded state. As such, when the system 3530 is deployed within a body vessel 3552, the valves 3636 extend from the intravascular device 3534 to the vessel wall 3554 (i.e., to the interior side 3538 of the tubular expandable body 3532) to cover a substantial portion of the flow area around the intravascular device 3534. The valves 3636 are shown being hingedly attached by hinges 3638 to the tubular expandable body 3532; however, any other suitable connection may be made between the hinges 3638 and the valves 3636.

The valves 3636 are configured to permit the flow of fluid in a forward direction through the valves 3636 and to resist the flow of fluid in a rearward direction through the valves 3636. In some embodiments, valves 3636 could be configured to prevent the flow of fluid in the rearward direction. Although four valves 3636 are illustrated in FIGS. 30A and 30B, it should be understood that merely one valve or any other suitable number of valves 3636 could be used. The valves 3636 could be of any suitable type, and are preferably similar to artificial or biological valves currently being used to repair human hearts. For example, the valves 3636 could be tilting disc valves, bileaflet valves, or ball valves, among others.

Further, the valves 3636 could be formed of any suitable material, such as a compliant material or a hard material. Suitable compliant materials include synthetic polyester, such as that manufactured under the name DACRON™, and biocompatible urethane. A suitable hard material is carbon composite.

For percutaneous delivery of the attachment system 3530, the valves 3636 may need to be bent or slit if the valves 3636 are formed of compliant material, or slit if formed of hard material. For example, the valves 3636 could be bent or slit along the lines 3640, and it is contemplated that many more bends or slits may be made in a single valve 3636, however, only one line 3640 is shown for clarity reasons.

Various embodiments of attachment systems have been described herein. It should be understood that the variations between the embodiments described could be used in other of the embodiments described. Further, the attachment systems of the present invention may be equipped with various features that enhance the ability to retrieve the devices comprising the systems. The systems may be configured to be permanent, retrievable, or partially retrievable (for an example of a partially retrievable system, refer to FIGS. 12A-12D).

Placement of each attachment system described herein may be accomplished by entry into the arterial or venous system through a variety of minimally invasive methods. The attachment systems described herein may be introduced percutaneously, for example, using the delivery system described in FIGS. 2A-3, or using any other suitable delivery system. Each intravascular device and its supporting fixture may be deployed in a single stage delivery or in a multi-stage delivery.

Some variations of the attachment systems described herein may also serve as an embolic filter, for example, refer to FIGS. 6A, 6B, 11A-15C, 17A-17B, 19-23,25, and 28-29B. This may be especially important if the attachment system is placed in the aorta.

Furthermore, each attachment system described herein may provide integral support for the function of the intravascular device. For example, the attachment system could serve as an antenna, as electrical contacts, as an electrical conduit, as an electrical insulator, as a heat sink or source, or to preferentially redirect or assist in fluid flow.

As a person skilled in the art will readily appreciate, the above description is meant as an illustration of implementation of the principles this invention. This description is not intended to limit the scope or application of this invention in that the invention is susceptible to modification, variation and change, without departing from the spirit of this invention, as defined in the following claims.

What is claimed is:

1. A method for attaching an intravascular device to a vessel wall of a body vessel, comprising:
introducing the intravascular device into the body vessel, the intravascular device comprising a cardiac assist device for assisting with blood circulation, a biological attachment material connected to the cardiac assist device;
allowing the biological attachment material to adhere to the vessel wall such that host cells of the vessel wall grow into the biological attachment material.

2. The method of claim 1, wherein the biological attachment material comprises an extracellular matrix material.

3. The method of claim 2, wherein the extracellular matrix material comprises small intestinal submucosa (SIS).

4. The method of claim 1, wherein the intravascular device further comprises a plurality of barbs connected to the cardiac assist device to anchor the cardiac assist device to the vessel wall.

5. The method of claim 4, wherein each barb is connected to the cardiac assist device with a biodegradable connection.

6. The method of claim 5, wherein the biodegradable connection is a biodegradable weld.

7. The method of claim 1, wherein the cardiac assist device comprises a pump and a pump housing.

8. The method of claim 7, wherein the cardiac assist device comprises a through channel to allow fluid to flow therethrough in a substantially unimpeded manner.

9. The method of claim 1, wherein the intravascular device further comprises a retrieval member to facilitate the removal of the intravascular device.

10. The method of claim 9, wherein the retrieval member is selected from the group consisting of a tether, an electrical conduit, and a drug delivery channel.

11. The method of claim 5, wherein the biodegradable connection comprises at least one of a magnesium alloy, silver, a polymer, and a degradable suture.
